(19) United States
(12) Patent Application Publication
(10) Pub. No.: US 2007/0292386 A9
(43) Pub. Date: Dec. 20, 2007

(54) Vaccine formulations for intradermal delivery comprising adjuvants and antigenic agents

(76) Inventors: Robert L. Campbell, Bahama, NC (US); Kevin G. Dolan, Holly Springs, NC (US); Wendy Woodley, Cary, NC (US)

Correspondence Address: BECTON, DICKINSON AND COMPANY;(ALSTON & BIRD LLP), 1 BECTON DRIVE, MC 110, FRANKLIN LAKES, NJ, 07417-1880, US

(21) Appl. No.: 11/292,659

(22) Filed: Dec. 2, 2005

Related U.S. Application Data
(60) Provisional application No. 60/632,569, filed on Dec. 2, 2004.

(51) Int. Cl. A61K 39/02; A61K 39/00; A61K 48/00; A61K 38/19

(52) U.S. Cl. 424/85.1; 424/234.1; 514/44; 424/274.1

(57) ABSTRACT

The present invention relates to compositions for intradermal delivery of an antigenic or immunogenic agent in combination with one or more adjuvants. The immunogenic compositions of the invention comprise an antigenic or immunogenic agent and at least one adjuvant, which enhances the immune response to the antigenic or immunogenic agent, once delivered to the intradermal compartment of a subject's skin. The immunogenic compositions of the invention have enhanced efficacy as the adjuvants of the composition promote recruitment of antigen presenting cells to the intradermal compartment and thus enhance presentation and/or availability of the antigenic or immunogenic agent to the antigen presenting cells. The enhanced efficacy of the immunogenic compositions of the invention results in a therapeutically and/or prophylactically effective immune response after a single intradermal dose, with lower doses of adjuvant than conventionally used, achieving therapeutic efficacy from a single administration.

This application claims the benefit of priority of U.S. Provisional Application No. 60/632,569 filed Dec. 2, 2004 which is incorporated herein by reference in its entirety.

### 1. FIELD OF THE INVENTION

The present invention relates to compositions for intradermal delivery of an antigenic or immunogenic agent in combination with one or more adjuvants. The immunogenic compositions of the invention comprise an antigenic or immunogenic agent and at least one adjuvant, which enhances the immune response to the antigenic or immunogenic agent, once delivered to the intradermal compartment of a subject's skin. The enhanced efficacy of the immunogenic compositions of the invention results in a therapeutically and/or prophylactically effective immune response after a single intradermal dose, with lower doses of adjuvant than conventionally used, and without the need for booster immunizations.

### 2. BACKGROUND OF THE INVENTION

Adjuvants are agents that enhance the efficacy and protective immune response of an immunogenic composition, e.g., vaccines. Traditionally, the immunogenicity of an immunogenic composition has been improved by adding an adjuvant to the composition. Immunological adjuvants were initially described by Ramon (1924, Ann. Inst. Pasteur, 38: 1) as “substances used in combination with a specific antigen that produced a more robust immune response than the antigen alone.”

A wide variety of substances, both biological and synthetic, have been used as adjuvants. However, despite extensive evaluation of a large number of candidates over many years, the only adjuvants currently approved by the U.S. Food and Drug administration are aluminum-based minerals (generically called Alum). Many experimental adjuvants have advanced to clinical trials since the development of Alum, and some have demonstrated high potency but have proven too toxic for therapeutic use in humans.

Furthermore, the efficacy of adjuvants varies depending on the target compartment in a subject for the delivery of immunogenic compositions, and thus each adjuvant must be validated according to the composition's contemplated target compartment. Whereas a number of adjuvants or potential adjuvants have been found and validated for spaces other than the intradermal compartment, e.g., intramuscular, intravenous or subcutaneous, prior to the instant invention there were few known adjuvants with efficacy in the intradermal compartment. Therefore, there is an unmet need for an adjuvant that can effectively enhance immune response triggered by an intradermally administered antigenic or immunogenic agent.

### 3. SUMMARY OF THE INVENTION

The present invention is based, in part, on the surprising discovery by the inventors of an intradermal vaccine delivery composition which enhances the therapeutic efficacy and immune response of the vaccine by specifically targeting the intradermal compartment of a subject's skin. Indeed, the intradermal compartment has rarely been effectively targeted as a site of delivery of an antigenic or immunogenic agent, at least, in part, due to the difficulty of a specific and reproducible delivery of the antigenic or immunogenic agent, i.e., the precise needle placement into the intradermal space and adequate pressures of delivery.

The benefits of the invention are also appreciated in other dermal compartments including but not limited to the epidermal compartment of skin.

The inventors have found that intradermal delivery of certain compounds, e.g., adjuvants, in combination with an antigenic or immunogenic agent results in an enhanced immune response to the antigenic or immunogenic agent. The compositions of the invention have enhanced efficacy, e.g., enhanced protective immune response, as delivery of the compound in combination with the antigenic or immunogenic agent to the intradermal compartment results in an enhanced availability and/or presentation of the antigenic or immunogenic agent to the antigen presenting cells that are recruited therein.

The enhanced efficacy of the compositions of the inventions may be achieved with dermal vaccine compositions including formulations for intradermal and epidermal delivery. In some embodiments, the dermal vaccine compositions of the invention (including the epidermal and intradermal compositions) comprise an antigenic or immunogenic agent, and at least one adjuvant, which enhances the presentation and/or availability of the antigenic or immunogenic agent to an immune cell, e.g., the immune cells of the intradermal compartment (e.g., antigen presenting cells) or the immune cells of the epidermal compartment (e.g., epidermal Langerhan's cells (LC)), resulting in an enhanced protective immune response. In a specific embodiment, the adjuvant acts to prolong the exposure of the antigenic or immunogenic agent to the immune cells of the dermal compartment, e.g., antigen presenting cells, epidermal Langerhan's cells (LC), resulting in an enhanced protective immune response.

The dermal vaccine compositions of the invention (including the epidermal and intradermal compositions) have enhanced efficacy, e.g., enhanced protective immune response, as the antigenic or immunogenic agent is delivered to the dermal compartment with an enhanced availability and/or presentation to the immune cells that reside therein, e.g., antigen presenting cells. Alternatively, the dermal vaccine compositions of the invention have enhanced efficacy as the antigenic or immunogenic agent is delivered to the dermal compartment, with a prolonged exposure of the antigenic or immunogenic agent to the immune cells that reside therein, resulting in an enhanced immune response. The enhanced efficacy of the dermal vaccine compositions (including the epidermal and intradermal compositions) results in a therapeutically and/or prophylactically effective response, e.g., protective immune response, after a single dermal dose, with lower doses of the antigenic or immunogenic agent than conventionally used, and without the need for booster immunizations. The dermal vaccine formulations of the invention are particularly effective as they also allow lower doses of adjuvants to be used relative to amounts used in conventional amounts in vaccine formulations, while resulting in the same or in an enhanced immune response.

The invention encompasses compositions for intradermal delivery comprising an antigenic or immunogenic agent, and at least one adjuvant, which enhances the immune response to the antigenic or immunogenic agent resulting in an enhanced immune response, preferably a protective immune response. In some embodiments, the adjuvants used in the immunogenic compositions of the invention allow the exposure of the antigenic or immunogenic agent to the immune cells of the intradermal compartment, by recruiting antigen presenting cells to the site of injection, resulting in an enhanced immune response to the antigenic or immunogenic agent.

Compounds that may be used in the immunogenic compositions of this invention include, but are not limited to, amorphous materials such as mineral salts, serum proteins, nucleic acids, cytokines, plant components such as saponins, bacterial and yeast antigens, and mammalian peptides. The invention particularly encompasses compounds or agents which have not been previously associated with an adjuvant activity in the intradermal compartment. The concentration of the adjuvant compound used in the immunogenic compositions of the invention depends on the particular compound used. In some embodiments, the concentration of the adjuvant compound used in the immunogenic compositions of the invention may be at least 0.01% (v/v), at least 0.1% (v/v), at least 1% (v/v), at least 10% (v/v) or in weight per volume terms from at least 0.001% w/v to at least 0.09% w/v. In specific embodiments, the concentration of the adjuvant compound used in the immunogenic compositions of the invention is from about 1.25 μg to about 2.5 μg per administration site. In some embodiments, the concentration at which the adjuvant compound is used results in mild skin irritation. In a preferred embodiment, if a compound results in skin irritation, one or more excipients may be added to the compositions to reduce or eliminate the irritation while maintaining the adjuvant activity of the compound in the immunogenic composition of the invention.

Antigenic or immunogenic agents that may be used in the immunogenic compositions of the invention include antigens from an animal, a plant, a bacteria, a protozoan, a parasite, a virus or a combination thereof. The antigenic or immunogenic agent may be any viral peptide, protein, polypeptide, or a fragment thereof derived from a virus including, but not limited to, RSV-viral proteins, e.g., RSV F glycoprotein, RSV G glycoprotein, influenza viral proteins, e.g., influenza virus neuramimidase, influenza virus hemagglutinin, herpes simplex viral protein, e.g., herpes simplex virus glycoprotein including for example, gB, gC, gD, and gE. The antigenic or immunogenic agent for use in the compositions of the invention may be an antigen of a pathogenic virus such as, an antigen of adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxyiridae (e.g., chordopoxyirinae, parapoxvirus, avipoxvirus, capripoxvirus, leporipoxvirus, suipoxvirus, molluscipoxvirus, and entomopoxyirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), metapneumovirus (e.g., avian pneumovirus and human metapneumovirus), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses), lentivirus (e.g. human immunodeficiency virus 1 and human immunodeficiency virus 2), spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Alternatively, the antigenic or immunogenic agent in the immunogenic compositions of the invention may be a cancer or tumor antigen including but not limited to, KS 1/4 pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MAA), prostate specific membrane antigen, carcinoembryonic antigen (CEA), polymorphic epithelial mucin antigen, human milk fat globule antigen, colorectal tumor-associated antigens such as: CEA, TAG-72, CO17-1A; GICA 19-9, CTA-1 and LEA, Burkitt's lymphoma antigen-38.13, CD19, human B-lymphoma antigen-CD20, CD33, melanoma specific antigens such as ganglioside GD2, ganglioside GD3, ganglioside GM2, ganglioside GM3, tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen, differentiation antigen such as human lung carcinoma antigen L6, L20, antigens of fibrosarcoma, human leukemia T cell antigen-Gp37, neoglycoprotein, sphingolipids, breast cancer antigen such as EGFR (Epidermal growth factor receptor), HER2 antigen (p185^{HER2}), polymorphic epithelial mucin (PEM), malignant human lymphocyte antigen-APO-1, differentiation antigen such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC 10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos, and T cell receptor derived peptide from a Cutaneous T cell Lymphoma.

The antigenic or immunogenic agent for use in the compositions of the invention may be any substance that under appropriate conditions results in an immune response in a subject, including, but not limited to, polypeptides, peptides, proteins, glycoproteins, lipids, nucleic acids, and polysaccharides. The concentration of the antigenic or immunogenic agent in the compositions of the invention may be determined using standard methods known to one skilled in the art and depends on the potency and nature of the antigenic or immunogenic agent. Given the enhanced delivery system of the invention, the concentration of the antigenic or immunogenic agent is preferably less than the conventional amounts used when alternative routes of administration are employed and alternative compositions.

In some embodiments, the immunogenic compositions of the invention further comprise one or more additives, including, but not limited to, excipients, stabilizers, and penetration enhancers.

The immunogenic compositions of the invention are particularly advantageous for developing rapid and high levels of immunity against the antigenic or immunogenic agent, against which an immune response is desired. The compositions of the invention can achieve a systemic immunity at a protective level with a low dose of the antigenic or immunogenic agent. In some embodiments, the compositions of the invention result in a protective immune response with a dose of the antigenic or immunogenic agent which is 90%, 80%, 70%, 60%, 50%, or 40% or less of the dose conventionally used for the antigenic or immunogenic agent in obtaining an effective immune response. In preferred embodiments, the compositions of the invention comprise a dose of the antigenic or immunogenic agent which is lower than the conventional dose used in the art, e.g., the dose recommended in the Physician's Desk Reference, utilizing the conventional modes of delivery, e.g., intramuscular and intravenous and the conventional compositions, i.e., in the absence of adjuvant compounds of the invention. Preferably, the compositions of the invention result in a therapeutically or prophylactically effective immune response after a single intradermal dose. The compositions of the invention may be administered intradermally for annual immunizations. In some embodiments, the compositions of the invention result in an enhanced immune response with a dose of the adjuvant which is 90%, 80%, 70%, 60%, 50% or 40% or less of the dose conventionally used.

The immunogenic compositions of the instant invention have an enhanced therapeutic efficacy, safety, and toxicity profile relative to currently available formulations. The benefits and advantages imparted by the compositions of the invention are, in part, due to the particular formulation and their utility in targeting the intradermal compartment of skin. Preferably, the compositions of the invention provide a greater and more durable protection, especially for high risk populations that do not respond well to immunization.

The invention encompasses a method for eliciting an enhanced immune response to an antigenic or immunogenic composition in a subject, preferably an animal, more preferably a human, comprising delivering an immunogenic composition into an intradermal compartment of the subject's skin, wherein the immunogenic composition comprises an antigenic or immunogenic agent and an adjuvant compound of the invention. In a specific embodiment, the immunogenic composition is a vaccine.

As used herein, and unless otherwise specified, the term “enhanced immune response” means that, when an antigenic or immunogenic agent of the invention is co-administered with one or more adjuvants of the invention, there is an increased antibody formation, measured using any standard methods known in the art and described in Section 5.4, below, in a subject that receives such an administration as compared to a subject to which same amount of the antigenic or immunogenic agent alone is administered. Preferably, an enhanced immune response means about 10%, 20%, 30%, 50%, 70%, or 100% or greater increase in antibody formation.

Alternatively, the term “enhanced immune response,” as used herein, means that, when an antigenic or immunogenic agent of the invention is co-administered with one or more adjuvant compounds of the invention, a smaller amount of the antigenic or immunogenic agent can be used to achieve the same level of antibody formation in a subject, as compared to a subject to which the antigenic or immunogenic agent alone is administered. Preferably, the antigenic or immunogenic compound in an amount of about 90%, 80%, 70%, 60%, 50%, 40%, 30% or less of the amount of the same agent administered without the adjuvant compounds of the invention, may be administered to achieve the same level of antibody formation in a subject when administered together with the adjuvant compound of the invention.

It will be appreciated by one skilled in the art that the principles set forth herein are also applicable for delivering vaccine compositions beyond the stratum corneum for deposition into the epidermal compartment of a subject's skin. Methods and devices for abrading the skin, and particularly, the stratum corneum of the skin are known in the art and encompassed in the present invention for depositing a substance into the epidermal compartment, such as those disclosed in Patent Application Publication Nos. US 2003/0191085 and US 2003/0093040, both of which are hereby incorporated by reference in their entireties.

The invention further encompasses kits comprising an intradermal administration device and an immunogenic composition of the invention as described herein. In some embodiments, the invention provides a pharmaceutical pack or kit comprising a composition of the invention. In a specific embodiment, the invention provides a kit comprising, one or more containers filled with one or more of the components of the compositions of the invention, e.g., an anitgenic or immunogenic agent, and/or an adjuvant compound. In another specific embodiment, the kit comprises two containers, one containing an anitgenic or immunogenic agent, and the other containing the adjuvant compound. Associated with such container(s) can be a notice in the form prescribed by a governmental agency regulating the manufacture, use or sale of pharmaceuticals or biological products, which notice reflects approval by the agency of manufacture, use or sale for human administration. The invention further contemplates kits comprising a dermal administration device and a dermal vaccine formulation of the invention as described herein. The invention further contemplates kits comprising an epidermal administration device and an epidermal vaccine formulation of the invention as described herein.

3.1 Definitions

As used herein, the term “adjuvant” refers to any compound that assists or modifies the action of an agent, including but not limited to immunological adjuvants, which increase or diversify the immune response to an antigen. The term also encompasses compounds which when added to an immunogenic or antigen agent, non-specifically enhance an immune response to the agent in the recipient host upon exposure to the mixture. Adjuvants includes compounds that “immunomodulate” the cytokine network, up-regulating the immune response. Concomitant with this immunomodulation there is also a selection of which T-cell, Th1 or Th2, will mount this immune response. Th1 responses will elicit complement fixing antibodies and strong delayed-type hypersensitivity reactions associated with IL-2, IL-12, and gamma-interferon. Induction of CTL response appears to be associated with a TH1 response. Th2 responses are associated with high levels of IgE, and the cytokines IL-4, IL-5, IL-6 and IL-10. The term adjuvants includes compounds which, when administered to an individual or tested in vitro, increase the immune response to an antigen in a subject to which the antigen is administered, or enhance certain activities of cells from the immune system. Some antigens are weakly immunogenic when administered alone or are toxic to a subject at concentrations that evoke useful immune responses in a subject. An adjuvant can enhance the immune response of the subject to the antigen by making the antigen more strongly immunogenic. The adjuvant effect can also result in the ability to administer a lower dose of antigen to achieve a useful immune response in a subject.

As used herein, the term “antigen” refers to a molecule which contains one or more epitopes capable of stimulating a host's immune system to make a cellular antigen-specific immune response when the antigen is presented in accordance with the present invention, or a humoral antibody response. An antigen may be capable of eliciting a cellular or humoral response by itself or when present in combination with another molecule. Normally, an epitope will include between about 3-15, preferably about 5-15, and more preferably about 7-15 amino acids. Epitopes of a given protein can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. The term “antigen” as used herein denotes both subunit antigens, i.e., antigens which are separate and discrete from a whole organism with which the antigen is associated in nature, as well as killed, attenuated or inactivated bacteria, viruses, parasites or other microbes. Similarly, an oligonucleotide or polynucleotide which expresses a therapeutic or immunogenic protein, or antigenic determinant in vivo, such as in gene therapy and nucleic acid immunization applications, is also included in the definition of antigen herein. Further, for purposes of the present invention, antigens can be derived from any of several known viruses, bacteria, parasites and fungi, as well as any of the various tumor antigens. Furthermore, for purposes of the present invention, an “antigen” refers to a protein which includes modifications, such as deletions, additions and substitutions (generally conservative in nature), to the native sequence, so long as the protein maintains the ability to elicit an immunological response. These modifications may be deliberate, as through site-directed mutagenesis, or may be accidental, such as through mutations of hosts which produce the antigens.

As used herein, the term “immunological response” or “immune response” to an antigen or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of endogenous cytokines, chemokines and other such molecules produced by activated T-cells and/or other white blood cells, including those derived from CD4+ and CD8+ T-cells.

As used herein, and unless otherwise specified, the term “enhanced immune response” means that, when an antigenic or immunogenic agent of the invention is co-administered with one or more adjuvants of the invention, there is an increased antibody formation, measured using any standard methods known in the art and described in Section 5.4, below, in a subject that receives such an administration as compared to a subject to which same amount of the antigenic or immunogenic agent alone is administered. Preferably, an enhanced immune response means about 10%, 20%, 30%, 50%, 70%, or 100% or greater increase in antibody formation.

Alternatively, the term “enhanced immune response,” as used herein, means that, when an antigenic or immunogenic agent of the invention is co-administered with one or more adjuvant compounds of the invention, a smaller amount of the antigenic or immunogenic agent can be used to achieve the same level of antibody formation in a subject, as compared to a subject to which the antigenic or immunogenic agent alone is administered. Preferably, the antigenic or immunogenic compound in an amount of about 90%, 80%, 70%, 60%, 50%, 40%, 30% or less of the amount of the same agent administered without the adjuvant compounds of the invention, may be administered to achieve the same level of antibody formation in a subject when administered together with the adjuvant compound of the invention.

### 4. BRIEF DESCRIPTION OF FIGURES

FIG. 1 NEEDLE DEVICE. An exploded, perspective illustration of a needle assembly designed according to this invention.

FIG. 2 NEEDLE DEVICE. A partial cross-sectional illustration of the embodiment in FIG. 1.

FIG. 3 NEEDLE DEVICE. Embodiment of FIG. 2 attached to a syringe body to form an injection device.

FIGS. 4A-B. MICROABRADER DEVICE

A. an elevated view of the handle end of a preferred embodiment

B. a side view of a preferred embodiment of a microabrader.

FIGS. 5A-B. MICROABRADER DEVICE

A. a transparent perspective view of the microabrader device of FIGS. 4A and 4B.

B. a cross sectional view of the microabrader device of FIG. 4B.

FIG. 6 MICROABRADER DEVICE. A side view of the abrading surface the microabrader device of FIGS. 4A-B, 5A-B on the skin of a subject.

FIGS. 7A-B MICROABRADER DEVICE

A. a perspective view of the abrading surface in the embodiment of FIG. 6.

B. a cross sectional side view of the abrader surface.

FIG. 8 MICROABRADER DEVICE. A bottom view of the abrader surface of the embodiment of FIG. 6.

FIG. 9 MICROABRADER DEVICE. A perspective view in partial cross section of abraded furrows of skin

### 5. DETAILED DESCRIPTION OF THE INVENTION

The invention encompasses immunogenic compositions for dermal delivery, particularly intradermal delivery, comprising an antigenic or immunogenic agent, and at least one adjuvant, which enhances the immune response to the antigenic or immunogenic agent resulting in an enhanced immune response. In some embodiments, the immunogenic compositions result in an enhanced immune response. Although not intending to be bound by a particular mechanism of action, when the adjuvants of the instant invention are administered at the concentrations and by the delivery routes in accordance with the methods of the invention, they exhibit non-specific adjuvant activity, i.e., not through a specific cellular receptor, but perhaps through promotion of mechanical damage, mild irritation, or stretching of the skin. Alternatively, although not intending to be bound by a particular mechanism of action, once the adjuvants are delivered to the intradermal compartment of a subject's skin, they may act as a skin irritant leading to the recruitment of antigen presenting cells to the intradermal compartment at the site of the injection, and thus enhance the immune response to the immunogenic composition. Preferably, adjuvants used in the methods and immunogenic compositions of the invention have not been previously associated with an adjuvant activity. Most preferably, adjuvants used in the methods and immunogenic compositions of the invention have not been previously associated with an adjuvant activity in the intradermal space particularly at the dosages described herein.

The invention encompasses dermal vaccine compositions designed for targeted delivery of the antigenic or immunogenic agent, preferably, selectively and specifically to a particular compartment of a subject's skin including the intradermal and epidermal compartments. In some embodiments, the intradermal vaccine formulations of the invention are targeted directly to the intradermal compartment of skin. The intradermal vaccine formulations of the invention comprise an antigenic or immunogenic agent and at least one adjuvant, which enhances the presentation and/or availability of the antigenic or immunogenic to the an immune cell, such as the immune cells of the intradermal compartment, resulting in an enhanced protective immune response. In a specific embodiment, the adjuvant in the intradermal vaccine formulations of the invention prolongs the exposure of the antigenic or immunogenic agent to the immune cells of the intradermal compartment, e.g., antigen presenting cells, resulting in an enhanced protective immune response.

Although not intending to be bound by a particular mechanism of action, some of the intradermal vaccine compositions of the invention achieve an enhanced therapeutic efficacy, e.g., enhanced protective immune response, in part, due to the persistence of the antigenic or immunogenic agent at the site of the injection, i.e., the “depot effect”. Preferably, the intradermal vaccine compositions of the invention decrease the clearance rate of the antigenic or immunogenic agent from the site of the injection. More preferably, the intradermal vaccine compositions of the invention allow slow release of the antigenic or immunogenic agent at the site of injection, e.g., the dermal space.

The intradermal vaccine compositions of the invention may enhance the immunological response or therapeutic efficacy of the antigenic or immunogenic agent by (1) enhancing the immunogenicity of the antigenic or immunogenic agent; (2) enhancing the speed and/or duration of the immune response; (3) modulating the avidity, specificity, isotype or class distribution of the antibody response; (4) stimulating cell-mediated immune response; (5) promoting mucosal immunity; or (6) decreasing the dose of the antigenic or immunogenic agent. Although not intending to be bound by a particular mode of action, the intradermal vaccine compositions of the invention enhance cell-mediated immune response by specifically targeting the antigenic or immunogenic agent to the intradermal compartment of skin, which comprises of antigen presenting cells, e.g., dendritic cells and Langerhan cells. The intradermal vaccine compositions of the invention may enhance cell-mediated and/or humoral mediated immune response. Cell-mediated immune responses that may be modulated by the intradermal vaccine compositions of the invention include for example, Th1 or Th2 CD4+ T-helper cell-mediated or CD8+ cytotoxic T-lymphocytes mediates responses.

In some embodiments, the dermal vaccine compositions of the invention are designed for targeted delivery of the antigenic or immunogenic agent, preferably, selectively and specifically, to the epidermal compartment of a subject's skin. In some embodiments, the epidermal vaccine compositions of the invention are targeted directly to the epidermal compartment of skin. The epidermal vaccine compositions of the invention comprise an antigenic or immunogenic agent and at least adjuvant, which enhances the presentation and/or availability of the antigenic or immunogenic to the an immune cell, such as the immune cells of the epidermal compartment, resulting in an enhanced protective immune response. In a specific embodiment, the adjuvant in the epidermal vaccine compositions of the invention prolongs the exposure of the antigenic or immunogenic agent to the immune cells of the epidermal compartment, e.g., antigen presenting cells, resulting in an enhanced protective immune response.

Compounds that may be used in the immunogenic compositions of this invention include, but are not limited to, amorphous materials such as mineral salts, serum proteins, nucleic acids, cytokines, plant components such as saponins, bacterial and yeast antigens, and mammalian peptides. The invention particularly encompasses compounds or agents which have not been previously associated with an adjuvant activity in the intradermal compartment. The concentration of the adjuvant compound used in the immunogenic compositions of the invention depends on the particular compound used. In some embodiments, the concentration of the adjuvant compound used in the immunogenic compositions of the invention may range from 0.01 to 10% v/v or 0.1 μg/mL to 1000 μg/mL. In some embodiments, where the adjuvant is a mineral salt, the concentration of the salt is 0.01 to 10% v/v, 0.01 to 1% v/v or 0.01 to 0.1% v/v. In other embodiments, where the adjuvant is a bacterial and yeast antigen the concentration of the adjuvants may be from 0.1-1000 μg/mL, 0.1 to 100 μg/mL, 0.1-10 μg/mL, or 0.1-1 μg/mL. In yet other embodiments, where the adjuvants is a plant component, serum protein, cytokine or a mammalian peptide the concentration may be 0.1 to 1000 μg/mL, 0.1 to 10 μg/mL or 0.1 to 1 μg/mL. In other embodiments, where the adjuvant is an immunostimulatory oligonucleotide, the concentration may be from about 0.9 μg/mL to 900 μg/mL or from about 90 ng to 90 μg total adjuvant per dose. In some embodiments, the concentration at which the adjuvant compound is used results in mild skin irritation. In a preferred embodiment, if a compound results in skin irritation, one or more excipients may be added to the compositions to reduce or eliminate the irritation while maintaining the adjuvant activity of the compound in the immunogenic composition of the invention.

Antigenic or immunogenic agents that may be used in the immunogenic compositions of the invention include antigens from an animal, a plant, a bacteria, a protozoan, a parasite, a virus or a combination thereof. The antigenic or immunogenic agent may be any viral peptide, protein, polypeptide, or a fragment thereof derived from a virus including, but not limited to, RSV-viral proteins, e.g., RSV F glycoprotein, RSV G glycoprotein, influenza viral proteins, e.g., influenza virus neuramimidase, influenza virus hemagglutinin, herpes simplex viral protein, e.g., herpes simplex virus glycoprotein including for example, gB, gC, gD, and gE. The antigenic or immunogenic agent for use in the compositions of the invention may be an antigen of a pathogenic virus such as, an antigen of adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxyiridae (e.g., chordopoxyirinae, parapoxvirus, avipoxvirus, capripoxvirus, leporipoxvirus, suipoxvirus, molluscipoxvirus, and entomopoxyirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), metapneumovirus (e.g., avian pneumovirus and human metapneumovirus), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses), lentivirus (e.g. human immunodeficiency virus 1 and human immunodeficiency virus 2), spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Alternatively, the antigenic or immunogenic agent in the immunogenic compositions of the invention may be a cancer or tumor antigen including but not limited to, KS 1/4 pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MAA), prostate specific membrane antigen, carcinoembryonic antigen (CEA), polymorphic epithelial mucin antigen, human milk fat globule antigen, colorectal tumor-associated antigens such as: CEA, TAG-72, CO17-1A; GICA 19-9, CTA-1 and LEA, Burkitt's lymphoma antigen-38.13, CD19, human B-lymphoma antigen-CD20, CD33, melanoma specific antigens such as ganglioside GD2, ganglioside GD3, ganglioside GM2, ganglioside GM3, tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen, differentiation antigen such as human lung carcinoma antigen L6, L20, antigens of fibrosarcoma, human leukemia T cell antigen-Gp37, neoglycoprotein, sphingolipids, breast cancer antigen such as EGFR (Epidermal growth factor receptor), HER2 antigen (p185^{HER2}), polymorphic epithelial mucin (PEM), malignant human lymphocyte antigen-APO-1, differentiation antigen such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, GM2, OFA-2, G_{D2}, and M1:22:25:8 found in embryonal carcinoma cells, and SSEA-3 and SSEA-4 found in 4 to 8-cell stage embryos, and T cell receptor derived peptide from a Cutaneous T cell Lymphoma.

In some embodiments, the dermal vaccine formulations of the invention (including intradermal and epidermal vaccine formulations) further comprise one or more additives including, but not limited to, an adjuvant, an excipient, a stabilizer, a penetration enhancer, and a muco or bioadhesive.

In other embodiments, the dermal immunogenic compositions of the present invention (including intradermal and epidermal vaccine compositions) may further comprise one or more other pharmaceutically acceptable carriers, including any suitable diluent or excipient. Preferably, the pharmaceutically acceptable carrier does not itself induce a physiological response, e.g., an immune response. Most preferably, the pharmaceutically acceptable carrier does not result in any adverse or undesired side effects and/or does not result in undue toxicity. Pharmaceutically acceptable carriers for use in the dermal vaccine formulations of the invention (including intradermal and epidermal vaccine formulations) include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition; all of which is incorporated herein by reference in its entirety).

In particular embodiments, the dermal immunogenic compositions of the invention (including intradermal and epidermal vaccine compositions), may also contain wetting agents, emulsifying agents, or pH buffering agents. The dermal immunogenic compositions of the invention (including intradermal and epidermal vaccine compositions) can be a solid, such as a lyophilized powder suitable for reconstitution, a liquid solution, a suspension, a tablet, a pill, a capsule, a sustained release formulation, or a powder. In a specific preferred embodiment, the intradermal vaccine composition of the invention is not an emulsion, since intradermal delivery of emulsions are technically difficult and are labor intensive.

The intradermal vaccine compositions of the invention may be in any form suitable for intradermal delivery. In one embodiment, the intradermal vaccine composition of the invention is in the form of a flowable, injectable medium, i.e., a low viscosity compositions that may be injected in a syringe. In another embodiment, the intradermal vaccine compositions of the invention is in the form of a gelatinous matrix, e.g., a semi-solid or solid two or three dimensional matrix. In yet another embodiment, the intradermal vaccine compositions of the invention is in the form of a highly viscous, thick medium with limited fluidity. In either embodiment, the antigenic or immunogenic agent is uniformly and homogenously dispersed throughout the compositions.

The epidermal vaccine compositions of the invention may be in any form suitable for epidermal delivery, such as those disclosed in Patent Application Publication Nos. US 2003/0191085 and US 2003/0093040, both of which are hereby incorporated by reference in their entireties.

Preferably, the dermal vaccine compositions of the invention (including the intradermal and epidermal vaccine compositions) are stable compositions, i.e., undergo minimal to no detectable level of degradation and/or aggregation of the antigenic or immunogenic agent, and can be stored for an extended period of time with no loss in biological activity, e.g., antigenicity or immunogenicity of the antigenic agent.

The concentration of the antigenic or immunogenic agent in the dermal vaccine composition of the invention (including intradermal and epidermal vaccine compositions) may be determined using standard methods skilled in the art and depends on the potency and nature of the antigenic or immunogenic agent. Given the enhanced delivery system of the invention, the concentration of the antigenic or immunogenic agent is preferably less than the conventional amounts used when alternative routes of administration are employed, e.g., intramuscular. The concentration of the antigenic or immunogenic agent used in the dermal vaccine compositions of the invention (including intradermal and epidermal vaccine compositions) is 60%, preferably 50%, more preferably 40% of the concentration conventionally used in obtaining an effective immune response. Typically, the starting concentration of the antigenic or immunogenic agent in the dermal vaccine compositions of the invention (including intradermal and epidermal vaccine compositions) is the amount that is conventionally used for eliciting the desired immune response, using the conventional routes of administration, e.g., intramuscular injection. The concentration of the antigenic or immunogenic agent in the dermal vaccine compositions of the invention (including intradermal and epidermal vaccine compositions) is then adjusted, e.g., by dilution using a suitable diluent, so that an effective protective immune response is achieved, as assessed using standard methods known in the art and described herein.

The concentration of the adjuvant compound used in the immunogenic compositions of the invention depends on the particular compound used. In some embodiments, the concentration of the adjuvant compound used in the immunogenic compositions of the invention may be at least 0.01% (w/v), at least 0.1% (w/v), at least 1% (w/v), at least 10% (w/v), at least 15% (w/v), at least 20% (w/v), at least 25% (w/v), or at least 30% (w/v). In some embodiments, the concentration of the adjuvant is greater than about 30% (w/v). In other embodiments, the concentration of the adjuvant compound is at least 0.1% (w/v), at least 0.5% (w/v), at least 1% (w/v), at least 5% (w/v), or at least 10% (w/v).

The dermal vaccine compositions of the present invention (including intradermal and epidermal vaccine compositions) can be prepared as unit dosage forms. A unit dosage per vial may contain 0.1 mL to 1 mL, preferably 0.1 to 0.5 mL of the compositions. In some embodiments, a unit dosage form of the dermal vaccine compositions of the invention may contain 50 μL to 100 μL, 50 μL to 200 μL, or 50 μL to 500 μL of the composition. If necessary, these preparations can be adjusted to a desired concentration by adding a sterile diluent to each vial. The dermal vaccine compositions of the invention are more effective in eliciting the desired immune response, and thus the total volume for dermal delivery may be less than the volume that is conventionally used.

In some embodiments, the components of the dermal vaccine compositions of the invention, e.g., the antigenic or immunogenic agent and the molecule, e.g., polymer, are supplied either separately or mixed together in unit dosage form, for example, as a dry lyophilized powder or water free concentrate in a hermetically sealed container such as an ampoule or a sachette indicating the quantity of the active agent, e.g., the antigenic or immunogenic agent. In other embodiments, an ampoule of sterile diluent can be provided so that the components may be mixed prior to administration. In a specific embodiment, the adjuvant may be mixed with the antigenic or immunogenic agent just prior to administration. In another specific embodiment, the adjuvant may be mixed with the antigenic or immunogenic agent in an dermal delivery device during administration. In another specific embodiment, the adjuvant may be mixed with the antigenic or immunogenic agent in a dermal delivery device during administration. In another specific embodiment, the molecule may be mixed with the antigenic or immunogenic agent in an epidermal delivery device during administration.

The invention also provides dermal vaccine compositions that are packaged in a hermetically sealed container such as an ampoule or a sachette indicating the quantity of the components. In one embodiment, the dermal vaccine composition is supplied as a liquid, in another embodiment, as a dry sterilized lyophilized powder or water free concentrate in a hermetically sealed container and can be reconstituted, e.g., with water or saline to the appropriate concentration for administration to a subject. In an alternative embodiment, the dermal vaccine composition is supplied in liquid form in a hermetically sealed container indicating the quantity and concentration of the components.

The intradermal immunogenic compositions including vaccine compositions of the invention have particular utility for intradermal delivery of the antigenic or immunogenic agent to the intradermal compartment of a subject's skin. Preferably, the intradermal immunogenic compositions of the invention are administered using any of the intradermal devices and methods disclosed in U.S. Pat. No. 6,494,865; Patent Application Publication Nos. US 2005/0096632, US 2002/0095134, US 2002/0156453, and US 2003/0100885; or International Publication No.'s EP 10922 444, published Apr. 18, 2001; WO 01/02178, published Jan. 10, 2002; and WO 02/02179, published Jan. 10, 2002; all of which are incorporated herein by reference in their entirety. The intradermal immunogenic compositions of the invention are administered to the intradermal compartment of a subject's skin such that the intradermal space of the subject's skin is penetrated, without passing through it. Preferably, the intradermal immunogenic compositions are administered to the intradermal space at a depth of about 1.0 to 3.0 mm, most preferably at a depth of 1.0 to 2.0 mm. The intradermal immunogenic compositions of the invention for intradermal delivery provide a pain-free and less invasive mode of administration as compared to conventional modes of administrations, e.g., intramuscular, for vaccine compositions, and therefore are more advantageous, for example, in terms of the subjects' compliance. The actual method by which the immunogenic composition of the invention are targeted to the intradermal space is not critical as long as it penetrates the skin of a subject to the desired targeted depth within the intradermal space without passing through it. The actual optimal penetration depth will vary depending on the thickness of the subject's skin. In most cases, skin is penetrated to a depth of about 0.5-2 mm. Regardless of the specific intradermal device and method of delivery, the intradermal delivery preferably targets the immunogenic composition of this invention to a depth of at least 0.3 mm, more preferably at least 0.5 mm up to a depth of no more than 2.0 mm, more preferably no more than 1.7 mm.

The epidermal immunogenic compositions of the invention including the vaccine compositions of the invention have particular utility for epidermal delivery of the antigenic or immunogenic agent to the epidermal compartment of a subject's skin. Preferably, the epidermal compositions of the invention are administered using any of the methods and devices disclosed in Patent Application Publication Nos. US 2003/0191085 and US 2003/0093040, both of which are hereby incorporated by reference in their entirety.

In some embodiments, the immunogenic compositions are delivered at a targeted depth just under the stratum corneum and encompassing the epidermis and upper dermis, e.g., about 0.025 mm to about 2.5 mm. In order to target specific cells in the skin, the preferred target depth depends on the particular cell being targeted and the thickness of the skin of the particular subject. For example, to target the Langerhans cells in the dermal space of human skin, delivery would need to encompass, at least, in part, the epidermal tissue depth typically ranging from about 0.025 mm to about 0.2 mm in humans.

In some embodiments, the dermal immunogenic compositions of the invention (including the intradermal and epidermal compositions) are administered within 12 hours, preferably within 6 hours, within 5 hours, within 3 hours, or within 1 hour after preparation, for example, after being reconstituted from the lyophilized powder. In a preferred embodiment, the dermal immunogenic compositions are prepared for dermal administration into a subject immediately prior to the dermal administration, i.e., mixed with the adjuvant.

The dermal vaccine immunogenic compositions of the invention (including the intradermal and epidermal compositions) have little or no short term and/or long term toxicity when administered in accordance with the methods of the invention. Most preferably, the dermal vaccine immunogenic compositions of the invention when intradermally administered have little or no adverse or undesired reaction at the site of the injection, e.g., skin irritation, swelling, rash, necrosis, skin sensitization. In yet other most preferred embodiments, the epidermal vaccine immunogenic compositions of the invention when epidermally administered have little or no adverse or undesired reaction at the site of the injection, e.g., skin irritation, swelling, rash, necrosis, skin sensitization.

The invention provides methods of treatment and prophylaxis which involve administering an immunogenic composition of the invention to a subject, preferably a mammal, and most preferably a human for treating, managing or ameliorating symptoms associated with a disease or disorder, especially an infectious disease or cancer. The subject is preferably a mammal such as a non-primate, e.g., cow, pig, horse, cat, dog, rat, mouse and a primate, e.g., a monkey such as a Cynomolgous monkey and a human. In a preferred embodiment, the subject is a human. Preferably, the immunogenic composition of the invention is a vaccine composition.

The invention encompasses a method for immunization and/or stimulating an immunological immune response in a subject comprising dermal delivery (including intradermal and epidermal delivery) of a single dose of an dermal immunogenic composition of the invention to a subject, preferably a human. In some embodiments, the invention encompasses one or more booster immunizations. The immunogenic composition of the invention is particularly effective in stimulating and/or up-regulating an antibody response to a level greater than that seen in conventional immunogenic compositions (such as vaccines) and administration schedules. The immunogenic compositions of the invention are particularly advantageous for developing rapid and high levels of immunity against the antigenic or immunogenic agent, against which an immune response is desired. The immunogenic compositions of the invention can achieve a systemic immunity at a protective level with a low dose of the antigenic or immunogenic agent. In some embodiments, the immunogenic compositions of the invention result in an enhanced immune response with a dose of the antigenic or immunogenic agent which is 60%, preferably 50%, more preferably 40% of the dose conventionally used for the antigenic or immunogenic agent in obtaining an effective immune response. In preferred embodiments, the immunogenic compositions of the invention comprise a dose of the antigenic or immunogenic agent which is lower than the conventional dose used in the art, e.g., the dose recommended in the Physician's Desk Reference, utilizing the conventional modes of delivery, e.g., intramuscular and subcutaneous and the conventional compositions, i.e., in the absence of adjuvants of the invention. Preferably, the immunogenic compositions of the invention result in a therapeutically or prophylactically effective immune response after a single intradermal dose. The immunogenic compositions of the invention may be administered intradermally for annual immunizations.

The immunogenic compositions of the instant invention have an enhanced therapeutic efficacy, safety, and toxicity profile relative to currently available compositions. The benefits and advantages imparted by the immunogenic compositions of the invention is, in part, due to the particular compositions and their utility in targeting the dermal compartment of skin. Preferably, the immunogenic compositions of the invention provide a greater and more durable protection, especially for high risk populations that do not respond well to immunization.

The invention encompasses methods for determining the efficacy of immunogenic compositions of the invention using any standard method known in the art or described herein. Assays for determining the efficacy of the immunogenic compositions of the invention may be in vitro based assays or in vivo based assays, including animal based assays. In some embodiments, the invention encompasses detecting and/or quantitating a humoral immune response against the antigenic or immunogenic agent of a composition of the invention in a sample, e.g., serum, obtained from a subject who has been administered an immunogenic composition of the invention. Preferably, the humoral immune response of the immunogenic compositions of the invention are compared to a control sample obtained from the same subject, who has been administered a control composition, e.g., a composition which simply comprises of the antigenic or immunogenic agent.

In other embodiments, the invention encompasses methods for determining the efficacy of the compositions of the invention by measuring cell-mediated immune response. Methods for measuring cell-mediated immune response are known to one skilled in the art and encompassed within the invention. In some embodiments, a T cell immune response may be measured for quantitating the immune response in a subject, for example by measuring cytokine production using common methods known to one skilled in the art including but not limited to ELISA from tissue culture supernatants, flow cytometry based intracellular cytokine staining of cells ex vivo or after an in vitro culture period, and cytokine bead array flow cytometry based assay. In yet other embodiments, the invention encompasses measuring T cell specific responses using common methods known in the art, including but not limited to chromium based release assay, flow cytometry based tetramer or dimer staining assay using known CTL epitopes.

The invention also provides a pharmaceutical pack or kit comprising an intradermal immunogenic composition, e.g., vaccine composition of the invention. In a specific embodiment the invention provides a kit comprising, one or more containers filled with one or more of the components of the intradermal vaccine composition of the invention, e.g., an anitgenic or immunogenic agent, an adjuvant. In another specific embodiment, the kit comprises two containers, one containing an anitgenic or immunogenic agent, and the other containing the adjuvant. Associated with such container(s) can be a notice in the form prescribed by a governmental agency regulating the manufacture, use or sale of pharmaceuticals or biological products, which notice reflects approval by the agency of manufacture, use or sale for human administration.

The invention further contemplates kits comprising an intradermal administration device and an intradermal vaccine composition of the invention as described herein. The invention further contemplates kits comprising a dermal administration device and a dermal vaccine composition of the invention as described herein. The invention further contemplates kits comprising an epidermal administration device and an epidermal vaccine composition of the invention as described herein.

It will be appreciated by one skilled in the art that the principles set forth herein are also applicable for delivering immunogenic compositions beyond the stratum corneum for deposition into the epidermal compartment of a subject's skin. Methods and devices for abrading the skin, and particularly, the stratum corneum of the skin are known in the art and encompassed in the present invention for depositing a substance into the epidermal compartment, such as those disclosed in Patent Application Publication Nos. US 2003/0191085 and US 2003/0093040, both of which are hereby incorporated by reference in their entireties.

5.1 Compositions of the Invention

The immunogenic compositions of the invention are designed for targeted delivery of an antigenic or immunogenic agent, preferably, selectively and specifically, to the dermal compartment of a subject's skin, including the epidermal and intradermal compartment. In some embodiments, the compositions of the invention are targeted directly to the intradermal compartment of skin. In other embodiments, the compositions of the invention are targeted directly to the epidermal compartment of skin.

The compositions of the invention comprise an antigenic or immunogenic agent and at least one adjuvant, which enhances the immune response to the antigenic or immunogenic agent. In the present invention, an enhanced immune response, when used in connection with an immunogenic composition containing one or more adjuvants of the invention as compared to the same composition without the adjuvants, includes an increased antibody formation in a subject to which the composition is administered. In addition, an enhanced immune response includes a situation where a reduced amount of the antigenic or immunogenic agent is used in combination with the adjuvants to achieve the same level of antibody formation obtained when the antigenic or immunogenic agent is used without the adjuvants.

Although not intending to be bound by a particular mechanism of action, the adjuvants used in the immunogenic compositions of the invention result in skin irritation at the site of injection, which in turn recruits antigen presenting cells to the intradermal compartment and allows exposure of the antigenic or immunogenic agents to the antigen presenting cells. The compositions of the invention may enhance cell-mediated and/or humoral mediated immune response. Cell-mediated immune responses that may be modulated by the dermal compositions of the invention include for example, Th1 or Th2 CD4+ T-helper cell-mediated or CD8+ cytotoxic T-lymphocytes mediates responses.

In specific embodiments, the formulations of the invention are not in solid dose forms, i.e., the formulations are in liquid dose forms. Solid dose forms excluded herein are for example fibers, spheres, tablets, discs, particles such as those disclosed in U.S. Pat. No. 6,586,006. In other specific embodiments, the formulations of the invention are not polymeric micro particles such as those disclosed in U.S. Publication No. 2003/0138453 or PCT/US99/17308. In more specific embodiments, the formulations of the invention are not micro particles comprising poly(lactide-co-glycolides) (PLG).

In yet other specific embodiments, the formulations of the invention are not emulsions, e.g., an emulsion comprising a metabolizable oil and an emulsifying agent. In other more specific embodiments, the formulations of the invention are not submicron emulsions such as those disclosed in U.S. Publication No. 2003/0138453, wherein the emulsion comprises and oil droplet emulsion comprising droplets ranging in size from about 10 nm to about 1000 nm.

In specific embodiments, the formulations of the invention are not liposomal, i.e., do not contain liposomes including unilamellar, multilamellar, plurilamellar vesicles. In specific embodiments, the antigenic or immunogenic agents of the formulations of the invention are not linked, either covalently or non-covalently to any liposome. In other specific embodiments, the adjuvants of the formulations of the invention are not linked, either covalently or non-covalently to any liposome.

In some embodiments, the compositions of the invention further comprise one or more additives including, but not limited to, an excipient, a stabilizer, a penetration enhancer, and a muco or bioadhesive. In other embodiments, the compositions of the present invention may further comprise one or more other pharmaceutically acceptable carriers, including any suitable diluent or excipient. Preferably, the pharmaceutically acceptable carrier does not itself induce a physiological response, e.g., an immune response. Most preferably, the pharmaceutically acceptable carrier does not result in any adverse or undesired side effects and/or does not result in undue toxicity. Pharmaceutically acceptable carriers for use in the compositions of the invention include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition; all of which is incorporated herein by reference in its entirety).

In particular embodiments, the immunogenic compositions of the invention, may also contain wetting agents, emulsifying agents, or pH buffering agents. The compositions of the invention can be a solid, such as a lyophilized powder suitable for reconstitution, a liquid solution, a suspension, a tablet, a pill, a capsule, a sustained release formulation, or a powder.

The compositions of the invention may be in any form suitable for dermal delivery (including intradermal and epidermal delivery). Preferably, the compositions of the invention are stable formulations, i.e., undergo minimal to no detectable level of degradation and/or aggregation of the antigenic or immunogenic agent, and can be stored for an extended periods of time with no loss in biological activity, e.g., antigenicity or immunogenicity of the antigenic or immunogenic agent.

The compositions of the present invention can be prepared as unit dosage forms. A unit dosage per vial may contain 0.1 mL to 1 mL, preferably 0.1 to 0.5 mL of the immunogenic composition. In some embodiments, a unit dosage form of the compositions of the invention may contain 50 μL to 100 μL, 50 μL to 200 μL, or 50 μL to 500 μL of the composition. If necessary, these preparations can be adjusted to a desired concentration by adding a sterile diluent to each vial. The compositions of the invention are more effective in eliciting the desired immune response, and thus the total volume for dermal delivery may be less than the volume that is conventionally used.

In some embodiments, the components of the compositions of the invention, e.g., the antigenic or immunogenic agent and the adjuvant, are supplied either separately or mixed together in unit dosage form, for example, as a dry lyophilized powder or water free concentrate in a hermetically sealed container such as an ampoule or a sachette indicating the quantity of the active agent, e.g., the antigenic or immunogenic agent. In other embodiments, an ampoule of sterile diluent can be provided so that the components may be mixed prior to administration. In a specific embodiment, the adjuvant compound may be mixed with the antigenic or immunogenic agent just prior to administration. In another specific embodiment, the adjuvant compound may be mixed with the antigenic or immunogenic agent in a dermal delivery device during administration.

The invention also provides compositions that are packaged in a hermetically sealed container such as an ampoule or a sachette indicating the quantity of the components. In one embodiment, the composition is supplied as a liquid, in another embodiment, as a dry sterilized lyophilized powder or water free concentrate in a hermetically sealed container and can be reconstituted, e.g., with water or saline to the appropriate concentration for administration to a subject. In an alternative embodiment, the composition is supplied in liquid form in a hermetically sealed container indicating the quantity and concentration of the components. The composition of the invention may be prepared by any method that results in a stable, sterile, injectable formulation.

The intradermal immunogenic compositions including vaccine compositions of the invention have particular utility for intradermal delivery of the antigenic or immunogenic agent to the intradermal compartment of a subject's skin. Preferably, the intradermal immunogenic compositions of the invention are administered using any of the intradermal devices and methods disclosed in U.S. Pat. No. 6,494,865; Patent Application Publication Nos. US 2005/0096632, US 2002/0095134, US 2002/0156453, and US 2003/0100885; or International Publication No.'s EP 10922 444, published Apr. 18, 2001; WO 01/02178, published Jan. 10, 2002; and WO 02/02179, published Jan. 10, 2002; all of which are incorporated herein by reference in their entirety. The intradermal immunogenic compositions of the invention are administered to the intradermal compartment of a subject's skin such that the intradermal space of the subject's skin is penetrated, without passing through it. Preferably, the intradermal immunogenic compositions are administered to the intradermal space at a depth of about 1.0 to 3.0 mm, most preferably at a depth of 1.0 to 2.0 mm. The intradermal immunogenic compositions of the invention for intradermal delivery provide a pain-free and less invasive mode of administration as compared to conventional modes of administrations, e.g., intramuscular, for vaccine compositions, and therefore are more advantageous, for example, in terms of the subjects' compliance. The actual method by which the immunogenic composition of the invention are targeted to the intradermal space is not critical as long as it penetrates the skin of a subject to the desired targeted depth within the intradermal space without passing through it. The actual optimal penetration depth will vary depending on the thickness of the subject's skin. In most cases, skin is penetrated to a depth of about 0.5-2 mm. Regardless of the specific intradermal device and method of delivery, the intradermal delivery preferably targets the immunogenic composition of this invention to a depth of at least 0.3 mm, more preferably at least 0.5 mm up to a depth of no more than 2.0 mm, more preferably no more than 1.7 mm.

The epidermal immunogenic compositions of the invention including the vaccine compositions of the invention have particular utility for epidermal delivery of the antigenic or immunogenic agent to the epidermal compartment of a subject's skin. Preferably, the epidermal compositions of the invention are administered using any of the methods and devices disclosed in Patent Application Publication Nos. US 2003/0191085 and US 2003/0093040, both of which are hereby incorporated by reference in their entirety.

In some embodiments, the immunogenic compositions are delivered at a targeted depth just under the stratum corneum and encompassing the epidermis and upper dermis, e.g., about 0.025 mm to about 2.5 mm. In order to target specific cells in the skin, the preferred target depth depends on the particular cell being targeted and the thickness of the skin of the particular subject. For example, to target the Langerhans cells in the dermal space of human skin, delivery would need to encompass, at least, in part, the epidermal tissue depth typically ranging from about 0.025 mm to about 0.2 mm in humans.

In some embodiments, the dermal immunogenic compositions of the invention (including the intradermal and epidermal compositions) are administered within 12 hours, preferably within 6 hours, within 5 hours, within 3 hours, or within 1 hour after preparation, for example, after being reconstituted from the lyophilized powder. In a preferred embodiment, the dermal immunogenic compositions are prepared for dermal administration into a subject immediately prior to the dermal administration, i.e., mixed with the adjuvant.

The dermal vaccine immunogenic compositions of the invention (including the intradermal and epidermal compositions) have little or no short term and/or long term toxicity when administered in accordance with the methods of the invention. Most preferably, the dermal vaccine immunogenic compositions of the invention when intradermally administered have little or no adverse or undesired reaction at the site of the injection, e.g., skin irritation, swelling, rash, necrosis, skin sensitization. In yet other most preferred embodiments, the epidermal vaccine immunogenic compositions of the invention when epidermally administered have little or no adverse or undesired reaction at the site of the injection, e.g., skin irritation, swelling, rash, necrosis, skin sensitization.

In some embodiments, the dermal vaccine formulations of the invention may comprise a penetration enhancer. As used herein, a “penetration enhancer” is any molecule that, when added to a dermal vaccine formulation of the invention, enables or enhances permeation of the immunogenic or antigenic agent across biological membranes, thereby increasing absorption of the immunogenic or antigenic agent. Non-limiting examples of penetration enhancers include, various molecular weight chitosans, such as chitosan and N,O-carboxymethyl chitosan; poly-L-arginines; fatty acids, such as lauric acid; bile salts such as deoxycholate, glycolate, cholate, taurocholate, taurodeoxycholate, and glycodeoxycholate; salts of fusidic acid such as taurodihydrofusidate; polyoxyethylenesorbitan such as Tween™ 20 and Tween™ 80; sodium lauryl sulfate; polyoxyethylene-9-lauryl ether (Laureth™9); EDTA; citric acid; salicylates; caprylic/capric glycerides; sodium caprylate; sodium caprate; sodium laurate; sodium glycyrrhetinate; dipotassium glycyrrhizinate; glycyrrhetinic acid hydrogen succinate, disodium salt (Carbenoxolone™); acylcarnitines such as palmitoylcarnitine; cyclodextrin; and phospholipids, such as lysophosphatidylcholine. Preferably, the penetration enhancer is selected from the group consisting of chitosan, fatty acids, polyethylene sorbitol and caprylic/capric glycerides.

The dermal vaccine compositions of the invention may also comprise other additives besides a penetration enhancer. For example, the intradermal formulation of the invention may comprise a protein stabilizer, e.g., trehalose, sucrose, glycine, mannitol, albumin, glycerol. In some embodiments, antigen-stabilizing solutes, typically protein-stabilizing solutes, are incorporated into the dermal vaccine composition of the invention. Although not intending to be bound by a particular mechanism of action, the use of protein-stabilizing solutes, such as sucrose, aids in protecting and/or stabilizing the antigenic or immunogenic agent in the dermal vaccine formulation of the invention (especially when the antigenic or immunogenic agent is a protein).

In other embodiments, the dermal immunogenic compositions of the inventions (including the epidermal and intradermal compositions) are dried compositions in particulate form (e.g., powder form) and can be prepared using any of the methods disclosed in U.S. Patent Publication No. 2003/0186271 (to Hwang et al., Entitled: Pharmaceutical Compositions in Particulate Form, Published on Oct. 2, 2003), which is incorporated herein by reference in its entirety. The most preferred formulations are those that can be packed as powder into a delivery device, then reconstituted and delivered simultaneously. In one specific embodiment, the dried compositions for use in the methods of the invention may be prepared using a method, comprising one or more of the following steps: atomizing a liquid formulation of comprising an immunogenic composition of the invention to produce an atomized formulation; freezing said atomized formulation to form solid particles; and drying said solid particles to produce dried particles (e.g., a powder). Preferably, said atomized formulation comprises droplets having a volume mean diameter (as defined by W. H. Finley, The mechanics of inhaled pharmaceutical aerosols: An introduction, Academic Press, London, UK (2001), which is incorporated herein by reference in its entirety) of between about 35 μm and about 300 μm, more preferably between about 50 μm and about 300 μm, and/or said powder comprises dried particles having a volume mean diameter of between about 35 um and about 3001 μm, more preferably between about 50 μm about 300 μm. Most preferably, these droplets or particles have a volume mean diameter of between about 50 μm and about 100 μm. In a preferred embodiment, at least about 50% of the dried particles have a volume diameter within about 80% of the mean; more preferably, at least about 50% of the dried particles have a volume diameter within about 60% of the mean. In a preferred embodiment, the powder comprises dried particles that have a mean aerodynamic diameter (as defined in W. H. Finley, supra) of between about 8 μm and about 140 μm, more preferably between about 8 μm and about 80 μm, still more preferably between about 20 μm and about 70 μm. This method, and compositions made by the method, are sometimes generally referred to herein as a “spray-freeze-dry” method or compositions. Spray-freeze-dry compositions are particularly preferred for use in accordance with the methods of the invention, as the immunogenic compositions of the invention may be provided in powder form, which is easily reconstitutable in pre-filled delivery devices (including epidermal and intradermal devices).

5.1.1 Adjuvants

The invention is based, in part, on the discovery by the inventors that dermal delivery (including intradermal and epidermal delivery) of an antigenic or immunogenic agent in combination with one or more adjuvants disclosed herein results in an enhanced immune response to the antigenic or immunogenic agent. Preferably, the adjuvants used in the compositions and methods of the invention have not been previously associated with an adjuvant activity in the intradermal compartment and/or epidermal compartment.

As used herein, when an adjuvant acts as an irritant, it causes a reversible inflammatory effect on skin tissue by chemical action at the site of contact and yet is not corrosive. Inflammatory effect at the site of injection involves an influx of blood at the site of injection and may be marked by swelling, redness, heat, and/or pain. One skilled in the art can determine if an excipient is a skin irritant using the methods disclosed in Code of Federal Regulation (Title 16, Vol. 2; 6 CFR 1500.41, which is incorporated herein by reference in its entirety). According to 6 CFR 1500.41, a chemical is a skin irritant if, when tested on the intact skin of albino rabbits by the methods of 16 CFR 1500.41 for four hours exposure or by other appropriate techniques, it results in an empirical score of five or more. Preferably, the adjuvant compounds used in the methods of the invention have a score of 5 or less, more preferably a score of 4 or less, and most preferably a score of 3 or less. When a compound of the invention is characterized as a skin irritant, one or more excipients that are not skin irritants may be used in the compositions to reduce the skin irritation.

In certain embodiments, this invention encompasses a method of eliciting an enhanced immune response from an immunogenic composition in a subject, comprising delivering the immunogenic composition into a dermal compartment of the subject's skin (including intradermal and epidermal compartment), wherein the immunogenic composition comprises an antigenic or immunogenic agent and an adjuvant of the invention. Examples of the adjuvants that may be used in the immunogenic compositions of the invention include, but are not limited to, amorphous materials such as mineral salts, serum proteins, nucleic acids, cytokines, plant components such as saponin-based compounds, bacterial and yeast antigens, and mammalian peptides.

Examples of mineral salts include, but are not limited to, aluminum salts, aluminum phosphate (e.g., HCI Biosector Elsenbakken 23, DK-3600 Fredrikssund, Denmark), calcium phosphate (e.g., Superfos Biosector Als Frydenlundsvej 30, 2950 Vedback, Denmark), aluminum hydroxide (Alhydrogel), aluminum hydroxide in combination with gamma insulin (Algammulin), amorphous aluminum hydroxyphosphate (Adju-Phos), and deoxycholic acid—aluminum hydroxide complex (DOC/Alum). In one specific embodiment, the concentration of aluminum phosphate in a composition of the invention is from about 0.01% to about 10% sediment or v/v, more preferably about 0.03% to about 1% sediment or v/v. In another embodiment, the concentration of calcium phosphate in a composition of the invention is from about 0.01% to about 10% sediment or v/v, more preferably about 0.5% to about 3% sediment or v/v. In specific embodiments, the concentration range of mineral salts in a composition of the invention is from about 0.03 to about 1% sediment or v/v. The percent sediment value used to describe mineral salt preparations can be determined by drawing the suspension into a capillary tube and allowing the volume to settle overnight. The volume in the capillary tube taken up by the settled mineral salt is divided by the total liquid volume to yield percent sediment. The process can be expedited by using a clinical capillary tube centrifuge or hematocrit centrifuge. In specific embodiments, when mineral salts are used in the formulations of the invention the formulations are not liposomal or emulsions.

The invention encompasses use of serum proteins for use in the immunogenic compositions of the invention including, but not limited to, complement factor C3d, which is a 16 amino acid peptide (See, e.g., Fearon et al., 1998, Semin. Immunol. 10: 355-61; Nagar et al., 1998, Science; 280(5367):1277-81, Ross et al. 2000, Nature Immunol., Vol. 1(2), each of which is incorporated herein by reference in its entirety). C3d is also available commercially (e.g., Sigma Chemical Company Cat. C 1547). In one embodiment, the concentration of C3d in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL. It will be appreciated by one skilled in the art that the optimal C3d sequence will depend on the species to which the composition of the invention is administered.

Examples of nucleic acids, preferably immunostimulatory oligonucleotides that may be used in the immunogenic compositions of the invention include, but are not limited to, CpG, polyadenylic acid/poly uriddenlic acid, and Loxorbine (7-allyl-8-oxoguanosine). The CpG sequences known in the art are encompassed herein, e.g., U.S. Pat. No. 6,406,705 which is incorporated herein by reference in its entirety. In a specific embodiment, when the compositions of the invention are for use in the epidermal compartment, the adjuvant in the composition does not contain a CpG motif.

In a specific embodiment, the immunostimulatory oligonucleotide comprises an unmethylated cytosine-guanosine dinucleotide motif known as the CpG motif. As used herein, the phrase “CpG motif” refers to a dinucleotide portion of an oligonucleotide which comprises a cytosine nucleotide followed by a guanosine nucleotide. Such oligonucleotides can be prepared using conventional oligonucleotide synthesis well known to the skilled artisan. In some embodiments, the oligonucleotides of the invention comprise a modified backbone, such as a phosphorothioate or peptide nucleic acid, so as to confer nuclease resistance to the oligonucleotide. Modified backbones are well known to those skilled in the art. Preferred peptide nucleic acids are described in detail in U.S. Pat. Nos. 5,821,060, 5,789,573, 5,736,392, and 5,721,102, Japanese Patent No. 10231290, European Patent No. 839,828, and PCT Publication Numbers WO 98/42735, WO 98/42876, WO 98/36098, WO 98/27105, WO 98/20162, WO 98/16550, WO 98/15648, WO 98/04571, WO 97/41150, WO 97/39024, and WO 97/38013, the disclosures of which are incorporated herein by reference in their entirety. The oligonucleotide used in the compositions of the invention preferably comprise between about 6 and about 100 nucleotides, more preferably between about 8 and about 50 nucleotides, most preferably between about 10 and about 40 nucleotides. In a specific embodiment, the disonucleotide is 20 nucleotides in length. In addition, the oligonucleotides of the invention can comprise substitutions of the sugar moieties and nitrogenous base moieties. The invention encompasses oligonucleotides known in the art such as those disclosed in, for example, Krieg et al., Proc. Natl. Acad. Sci. USA, 1998, 95, 12631-12636, Klinman et al., Proc. Natl. Acad. Sci. USA, 1996, 93, 2879-2883, Weiner et al, Proc. Natl. Acad. Sci. USA, 1997, 94, 10833-10837, Chu et al., J. Exp. Med., 1997, 186, 1623-1631, Brazolot-Millan et al., Proc. Natl. Acad. Sci. USA, 1998, 95, 15553-15558, Ballas et al., J. Immunol., 1996, 157, 1840-1845, Cowdery et al., J. Immunol., 1996, 156, 4570-4575, Halpern et al., Cell Immunol., 1996, 167, 72-78, Yamamoto et al., Jpn. J. Cancer Res., 1988, 79, 866-873, Stacey et al., J. Immunol., 1996, 157, 2116-2122, Messina et al., J. Immunol., 1991, 147, 1759-1764, Yi et al., J. Immunol., 1996, 157, 4918-4925, Yi et al., J. Immunol., 1996, 157, 5394-5402, Yi et al., J. Immunol., 1998, 160, 4755-4761, Roman et al., Nat. Med., 1997, 3, 849-854, Davis et al., J. Immunol., 1998, 160, 870-876, Lipford et al., Eur. J. Immunol., 1997, 27, 2340-2344, Moldoveanu et al., Vaccine, 1988, 16, 1216-1224, Yi et al., J. Immunol., 1998, 160, 5898-5906, PCT Publication WO 96/02555, PCT Publication WO 98/16247, PCT Publication WO 98/18810, PCT Publication WO 98/40100, PCT Publication WO 98/55495, PCT Publication WO 98/37919, and PCT Publication WO 98/52581, the disclosures of which are incorporated herein by reference in their entirety.

It will be appreciated by one skilled in the art that the oligonucleotides of the invention comprise at least one CpG motif but can contain a plurality of CpG motifs. Preferred oligonucleotides comprise nucleotide sequences such as, 5′-CsAsTsgs AsCsgsCsCsTsgsAsCsgsTst-3′ It will be appreciated by one skilled in the art that the optimal sequence will depend on the species to which the composition is administered. In one specific embodiment, the concentration of CpG in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL.

Examples of cytokines that may be used in the compositions of the invention include, but are not limited to, interferons (e.g., interferon-gamma), interleukins (e.g., interleukin-2 (IL-2), interleukin-4 (IL-4), interleukin-6 (IL-6), interleukin-7 (IL-7), interleukin-12 (IL-12), interleukin-15 (IL-15)), colony stimulating factors, e.g., macrophage colony stimulating factors (M-CSF); G-CSF, GM-CSF), tumor necrosis factor (TNF), IL-1 and MIP-3a. Cytokines used in the instant invention have adjuvant activity when delivered to the dermal compartment including intradermal and epidermal compartments. The inventors have discovered a new use for cytokines, including fragments thereof which share the same biological activity of the full-length proteins as well as the DNA sequences which encode cytokines or fragments thereof. Cytokines disclosed herein and biologically active fragments thereof and alternatively the “naked” or transduced DNA encoding the cytokines or fragments thereof can be employed as an adjuvant, particularly for vaccines. In a specific embodiment, the cytokine used in the compositions of the invention is not IL-12. In yet another specific embodiment, the cytokine used in the compositions of the invention is not GM-CSF or IFN-γ.

A broad range of biological activities have been attributed to Interferon-gamma (IFN-γ), an interferon produced by lymphocytes activated by specific antigens or mitogens. IFN-γ, in addition to having antiviral activity, has important immunoregulatory functions. It is a potent activator of macrophages, has antiproliferative effects on transformed cells and can potentiate the antiviral and antitumor effects of the type I interferons. The isolation and characterization of a recombinant plasmid containing a cDNA sequence for human IFN-γ was reported by Gray et al. (1982, Nature: 295: 503-81 which is incorporated herein by reference in its entirety). Additionally, IFNs are commercially available, for example, from ICN Chemical Company (CAT# 195769). The invention encompasses IFN-γ, fragments thereof which share the same biological activity of the full-length IFN-γ, as well as the DNA sequences which encode IFN-γ or fragments thereof. In one embodiment of the invention, the concentration of interferon-gamma in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL.

Interleukin 2 (IL-2) is a potent immune stimulator, activating diverse cells of the immune system, including T cells, B cells, and monocytes. IL-2 is the main growth factor of T lymphocytes (Theze et al. 1996, Immunol. Today 17:481-486). By regulating T helper lymphocyte activity IL-2 increases the humoral and cellular immune responses. By stimulating cytotoxic CD8 T cells and NK cells this cytokine participates in the defense mechanisms against tumors and viral infections. A cDNA coding for human interleukin-2 (IL-2) has been cloned from a cDNA library prepared from partially purified IL-2 mRNA. The DNA sequence codes for a polypeptide which consists of 153 amino acids including a putative signal sequence (see, Taniguchi et al., 1983, Nature, 24-30; 302(5906):305-10; which is incorporated herein by reference in its entirety). Human IL-2 is composed of four a helices connected by loops of various length; as established by its three dimensional structure (see, MacKay, 1992, Science 257:410-413; which is incorporated herein by reference in its entirety). The invention encompasses IL-2, fragments thereof which share the same biological activity of the full-length IL-2, as well as the DNA sequences which encode IL-2 or fragments thereof. In one embodiment, the concentration of IL-2 in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL.

Interleukin-4 (IL-4) is a highly pleitropic lymphokine, exhibiting both stimulatory and inhibitory effects on the growth, differentiation, and functional activity of B and T-lymphocytes, myeloid cells, and cells of non-hemopoietic origin (see reviews by Trotta, 1991, Am. J. Reprod. Immuno. 25: 124-128; Paul, 1991, Blood, 77: 1859-1870; Miyajima et al., 1988, FASEB J 2: 2462-2473; each of which is incorporated herein by reference in its entirety). The mature protein sequence of recombinant human IL-4 contains 129 amino acids, including two potential sites for glycosylation. The amino acid sequences as deduced from the cDNAs have been reported for human (Yokota et al., 1986, Proc. Natl. Acad. Sci. USA 83: 5894-6; which is incorporated herein by reference in its entirety), murine (Lee et al., 1986 Proc. Natl. Acad. Sci. USA 83: 2061-5; Noma et al., 1986, Nature, 319: 640-6; each of which is incorporated herein by reference in its entirety) and rat (McKnight et al., 1991, Eur. J. Immunol. 21: 1187-1194; which is incorporated herein by reference in its entirety). The invention encompasses IL-4, fragments thereof which share the same biological activity of the full-length IL-4, as well as the DNA sequences which encode IL-4 or fragments thereof. In a specific embodiment, the concentration of IL-4 in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL.

The interleukin 6 (IL-6) cytokine family, which includes IL-6, leukemia inhibitory factor (LIF), oncostatin M (OSM), ciliary neurotrophic factor (CNTF), IL-11 and cardiotrophin-1 (CT-1), exhibits pleiotropy and redundancy in biological activities. The IL-6 family cytokines exhibit a helical structure. Their receptors belong to the type 1 cytokine receptor family. The receptors of the IL-6 family cytokines share a receptor subunit, which explains one of the mechanisms of functional redundancy. (For review, see Hibi et al., 1996, J. Mol Med. 74(1):1-12). Interleukin-6 (IL-6) is a multifunctional cytokine that plays a central role in host defense due to its wide range of immune and hematopoietic activities and its potent ability to induce the acute phase response. Overexpression of IL-6 has been implicated in the pathology of a number of diseases including multiple myeloma, rheumatoid arthritis, Castleman's disease, psoriasis, and post-menopausal osteoporosis (For review see, Simpson et al., 1997, Protein Sci. 6(5):929-55). The invention encompasses any member of the IL-6 family, fragments thereof which share the same biological activity of the full-length IL-6, as well as the DNA sequences which encode IL-6 or fragments thereof. In a specific embodiment, the concentration of IL-6 in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL.

IL-7, previously known as pre-B-cell growth factor and lymphopoietin-1, was originally purified on the basis of its ability to promote the proliferation of precursor B-cells. It has now been shown that IL-7 can also stimulate the proliferation of thymocytes, T cell progenitors and mature CD4+ and CD8+ T cells. IL-7 can induce the formation of lymphokine-activated killer (LAK) cells as well as the development of cytotoxic T lymphocytes (CTL). Among myeloid lineage cells, IL-7 can up-regulate the production of pro-inflammatory cytokines and stimulate the tumorocidal activity of monocytes/macrophages. IL-7 is expressed by adherent stromal cells from various tissues. Mouse IL-7 cDNA encodes a precursor protein of 154 amino residues containing a 25 amino acid residue signal peptide (mouse mRNA sequence has GENBANK Accession No. NM_{—}008371). A DNA sequence encoding the mature mouse IL-7 protein was expressed in E. coli (Namen, A. et al., 1988, Nature 333:571-573, which is incorporated herein by reference in its entirety). Human IL-7 has approximately 65% amino acid sequence identity with mouse IL-7 and both proteins exhibit cross-species activity. IL-7 bioactivities are mediated by the binding of IL-7 to functional high-affinity receptor complexes. The ligand binding subunit (IL-7 R) of the IL-7 receptor complex has been cloned from human and mouse sources. In addition to the membrane-anchored form of the IL-7 receptor, a human cDNA clone that encodes a soluble form of the IL-7 R has also been isolated (for a review of IL-7 biology see, Appasamy P M et al., 1993 Cancer Invest. 11(4):487-99, which is incorporated herein by reference in its entirety). The invention encompasses IL-7, fragments thereof which share the same biological activity of the full-length IL-7, as well as the DNA sequences which encode IL-7 or fragments thereof. In a specific embodiment, the concentration of IL-7 in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL.

Interleukin-12 (IL-12), originally called natural killer cell stimulatory factor, is a heterodimeric cytokine (e.g., M. Kobayashi et al., J. Exp. Med., 1709:827 (1989), which is incorporated herein by reference in its entirety). The expression and isolation of IL-12 protein in recombinant host cells is described in detail in International Patent Application WO90/05147, published May 17, 1990 (also European patent application No. 441,900), each of which is incorporated herein by reference in its entirety. The DNA and amino acid sequences of the 30 kd and 40 kd subunits of the heterodimeric human IL-12 are provided in the above recited international application. Research quantities of recombinant human and murine IL-12 are also available from Genetics Institute, Inc., Cambridge, Mass. IL-12 has been found to stimulate IFN-gamma production by NK cells and T cells (Chan et al., 1991 J. Exp. Med., 173:869). Therapeutic effects of IL-12 administered systemically have been reported (See, e.g., F. P. Heinzel et al., 1993 J. Exp. Med., 177:1505). The invention encompasses IL-12 protein, fragments thereof or DNA encoding IL-12 or fragments thereof in the compositions of the invention. In a specific embodiment, the concentration of IL-12 in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL.

Interleukin 15 (IL-15) is a novel cytokine whose biological activities are similar to those of IL-2 as it regulates T and natural killer cell activation and proliferation. Both IL-15 and IL-2 are found to bind common hematopoietin receptor subunits, and may compete for the same receptor, and thus negatively regulate each other's activity. The number of CD8+ memory cells is shown to be controlled by a balance between IL-15 and IL2. IL-1 Sinduces the activation of JAK kinases, as well as the phosphorylation and activation of transcription activators STAT3, STAT5, and STAT6. Studies of the mouse counterpart suggested that this cytokine may increase the expression of apoptosis inhibitor BCL2L1/BCL-x(L), possibly through the transcription activation activity of STAT6, and thus prevent apoptosis. IL-15 is identified by Human Genbank Accession No CAA71044, which is incorporated herein by reference. See also, Krause et al., 1996, Cytokine, 9:667-74; Grabstein et al., 1994 Science 264:965; Burton et al. 1994, Proc. Natl. Acad. Aci. USA 91:4935; Anderson, et al. 1995 Genomics 25:701; Bamford et al., 1995 Cytokine 7:595; each of which is incorporated herein by reference in their entireties. The invention encompasses IL-15 protein, fragments thereof or DNA encoding IL-15 or fragments thereof in the compositions of the invention. In a particular embodiment, the concentration of IL-15 in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL.

Macrophage inflammatory proteins (MIPs) are proteins that are produced by certain mammalian cells, for example, macrophages and lymphocytes, in response to stimuli, such as gram-negative bacteria, lipopolysaccharide and concanavalin A. The invention encompasses MIPs, fragments thereof, and DNAs encoding full length MIPs or fragments thereof. In particular, the invention encompasses Macrophage inflammatory protein 3 (MIP-3) also known as Small inducible cytokine A23 precursor (CCL23) (Myeloid progenitor inhibitory factor-1); having Genbank Accession No. P55773 and ATCC Deposit No. 75676 on Feb. 9, 1994 which has been characterized and disclosed in U.S. Pat. No. 5,504,003 (which is incorporated herein by reference in its entirety). See also, Patel et al., 1997, Exp Med. 185(7): 1163-72; Yoon et al., 1998, Blood, 91(9):3118-26; Rajarathnam et al., 2001, J Biol. Chem. 276(7):4909-16; each of which is incorporated herein by reference in their entireties. The invention encompasses MIP-3 protein, fragments thereof or DNA encoding MIP-3 or fragments thereof in the compositions of the invention. In a specific embodiment, the concentration of MIP-3a in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL.

The invention encompasses use of saponins in the immunogenic compositions of the invention. “Saponin,” as the term is used herein, encompasses natural and synthetic glycosidic triterpenoid compounds and pharmaceutically acceptable salts, derivatives, mimetics (e.g., isotucaresol and its derivatives) and/or biologically active fragments thereof, which possess adjuvant activity. In one illustrative embodiment, saponins employed in the compositions of the present invention can be purified from Quillaja saponaria Molina bark, as described in U.S. Pat. No. 5,057,540, the disclosure of which is incorporated herein by reference in its entirety. The adjuvant properties of saponins were first recognized in France in the 1930's. (see, Bomford et al., Vaccine 1992, 10: 572-577). Two decades later the saponin from the bark of the Quillaja saponaria Molina tree found wide application in veterinary medicine, but the variability and toxicity of these crude preparations precluded their use in human vaccines. (See, Kensil et al., In Vaccine Design: The Subunit and Adjuvant Approach; Powell, M. F., Newman, J. J., Eds.; Plenum Press: New York, 1995 pp. 525-541, which is incorporated herein by reference in its entirety). In the 1970's a partially purified saponin fraction known as Quil A was shown to give reduced local reactions and increased potency (see, Kensil et al., 1995). Further fractionation of Quil A, which consisted of at least 24 compounds by HPLC, demonstrated that the four most prevalent saponins, QS-7, QS-17, QS-18, and QS-21, were potent adjuvants (see, Kensil, C. R. Crit Rev. Ther. Drug Carrier Syst. 1996, 13, 1-55, which is incorporated herein by reference in its entirety; Kensil et al., 1995). QS-21 and QS-7 were the least toxic of these. Partly because of its reduced toxicity, highly purified state (though still a mixture of no less than four compounds), (see, Soltysik, S.; Bedore, D. A.; Kensil, C. R. Ann. N.Y. Acad. Sci. 1993, 690: 392-395, which is incorporated herein by reference in its entirety) and more complete structural characterization, QS-21 (3) was the first saponin selected to enter human clinical trials. (see, Kensil, 1996; Kensil, 1995).

Although not intending to be bound by a particular mechanism of action QS-21 and other Quillaja saponins increase specific immune responses to both soluble T dependent and T-independent antigens, promoting an Ig subclass switch in B-cells from predominantly IgG1 or IgM to the IgG2a and IgG2b subclasses (Kensil et al., 1995). The IgG2a and IgG2b isotypes are thought to be involved in antibody dependent cellular cytotoxicity and complement fixation (Snapper and Finkelman, In Fundamental Immunology, 4th ed.; Paul, W. E., Ed.: Lippincott-Raven: Philadelphia, Pa., 1999, pp. 831-861). These antibody isotypes also correlate with a Th-1 type response and the induction of IL-2 and IFN-.gamma.-cytokines which play a role in CTL differentiation and maturation (Constant and Bottomly, Annu. Rev. Immunology 1997, 15: 297-322). As a result, QS-21 and other Quillaja saponins are potent inducers of class I MHC-restricted CD8+ CTLs to subunit antigens (Kensil, 1996; Kensil et al., 1995).

According to an aspect of the present invention, a saponin employed in a composition of the invention comprises a Quillaja saponin. In one preferred embodiment of this aspect of the invention, the Quillaja saponin comprises QS-7, QS-17, QS-18 and/or QS-21. Examples of saponins that may be used in the compositions of the invention include, but are not limited to, purified Quil-A (see, e.g., U.S. Pat. No. 5,057,540; which is incorporated herein by reference in its entirety), GSK-1 (ginseng saponin), QS-21, Immune Stimulating Complexes (ISCOMS) (i.e., mix of saponins, cholesterol, phospholipid, and optionally surfactants; ISCOMS are particulate structures comprising fractions of Quil A and are haemolytic, see, e.g., EP 0 109 942 B1; WO 96/11711; WO 96/33739; each of which is incorporated herein by reference in its entirety), Iscoprep 7.0.3 (i.e., complex of saponin derivatives), and SBAS-2 (i.e., mix of QS-21 and MPL-A). In one embodiment, the concentration of Quil-A in a composition of the invention is from about 0.01 μg/mL to about 200 μg/mL, preferably about 0.1 μg/mL to about 100 μg/mL, preferably about 1 μg/mL to about 50 μg/mL, more preferably about 5 μg/mL to about 20 μg/mL. In a specific embodiment, wherein the adjuvant in the compositions of the invention is a saponin, such formulations are not liposomal. In another specific embodiment, wherein the adjuvant in the compositions of the invention is a saponin, such formulations are are not emulsions. In yet another specific embodiment, wherein the adjuvant in the compositions of the invention is a saponin, the antigenic agent is not fused to a heterologous sequence. In yet another specific embodiment, wherein the adjuvant in the compositions of the invention is a saponin, the composition does not further contain an immunostimulatory oligonucleotide containing CpG motifs. In yet another specific embodiment, wherein the adjuvant in the compositions of the invention is a saponin, the composition does not further contain an aminoalkyglucosaminide (AGP) molecule such as those disclosed in U.S. Publication No. 2003/019033. In yet another specific embodiment, wherein the adjuvant in the compositions of the invention is a saponin, saponin molecule is not linked to a lipophile (e.g., lipid, fatty acid, polyethylene glycol, terpene) such as those disclosed in U.S. Pat. No. 6,080,725.

Examples of bacterial or yeast antigens that may be used in the compositions of the invention include, but are not limited to: muramyl peptides such as, but not limited to, Immther™, theramide (MDP derivative), DTP-N-GDP, GMDP (GERBU adjuvant), MPC-026, MTP-PE, murametide, murapalmitine; MPL derivatives such as, but not limited to, MPL-A, MPL-SE, 3D-MLA, and SBAS-2 (i.e., mix of QS-21 and MPL-A); and mannon. Other muramyl peptides that may be used in the compositions of the invention include, but are not limited to, N-acetyl-muramyl-L-threonyl-D-isoglutamine (thr-MDP), N-acteyl-normuramyl-L-alanyl-D-isogluatme (nor-MDP), N-acetylmuramyl-L-alanyl-D-isogluatminyl-L-alanine-2-(1′-2′-dipalmitoyl-s-n-glycero-3-huydroxyphosphoryloxy)-ethylamine (MTP-PE). Such agents are commercially available, for example, MPL-A may be obtained from ICN Chemical Company (Cat # 150012) and Immther™ may be obtained from Dor Pharma Inc. In a specific embodiment, the bacterial antigen is not the heat labile entereotoxin of E. Coli.

In one specific embodiment, the concentration of Immther™ in a composition of the invention is from about 0.1 μg/mL to about 100 μg/mL, preferably about 1 μg/mL to about 50 μg/mL, more preferably about 5 μg/mL to about 20 μg/mL.

In another embodiment, the concentration of MPL-A in a composition of the invention is from about 0.1 μg/mL to about 1000 μg/mL, preferably about 1 μg/mL to about 500 μg/mL, more preferably about 10 μg/mL to about 20 μg/mL.

In another embodiment, the concentration of mannon in a composition of the invention is from about 0.1 μg/mL to about 100 μg/mL, preferably about 1 μg/mL to about 50 μg/mL, more preferably about 5 μg/mL to about 20 μg/mL.

Examples of mammalian peptides that may be used in the compositions of the invention include, but are not limited to, melanonin peptide 946, neutrophil chemo-attractant peptide, and elastin repeating peptide. See, e.g., Senior et al., 1984, J Cell Bio 99 (Elastin); Needle et al., 1979, J. Biol. Chem. 254 (Neutrophil); and (Peptide 946) Cox et al. 1994, Science, 264), each of which is incorporated herein by reference in its entirety. An exemplary sequence for elastin may comprise the following sequence: Val-Gly-Val-Ala-Pro-Gly. An exemplary sequence for neutrophil peptide may comprise the following: N-formyl-NIe Leu-Phe-NIe-Tyr-Lys. An exemplary sequence for Peptide 946 may comprise the following: Tyr-Leu-Glu-Pro-Gly-Pro-Val-Thr-Ala. In one embodiment, the concentration of melanonin peptide 946 in a composition of the invention is from about 0.01 μg/ml to about 200 μg/ml, preferably about 0.1 μg/ml to about 100 μg/ml, preferably about 1 μg/ml to about 50 μg/ml, more preferably about 5 μg/ml to about 20 μg/ml. In another embodiment, the concentration of neutrophil chemo-attractant peptide in a composition of the invention is from about 0.01 μg/ml to about 200 μg/ml, preferably about 0.1 μg/ml to about 100 μg/ml, preferably about 1 μg/ml to about 50 μg/ml, more preferably about 5 μg/ml to about 20 μg/ml. In yet another embodiment, the concentration of elastin repeating peptide in a composition of the invention is from about 0.01 μg/ml to about 200 μg/ml, preferably about 0.1 μg/ml to about 100 μg/ml, preferably about 1 μg/ml to about 50 μg/ml, more preferably about 5 μg/ml to about 20 μg/ml.

Adjuvants described herein are commercially available, or can be obtained using conventional methods well-known in the art. The adjuvants used in the compositions of the invention can exist in a liquid, gas or solid form. Further, it will be readily apparent to those of ordinary skill in the art that these compounds can be used alone or in combination with other adjuvants known in the art or described herein. Particularly, two or more adjuvants can be used in combination to achieve an additive or a synergistic effect.

5.1.2 Immunogenic or Antigenic Agents

Antigenic or immunogenic agents that may be used in the immunogenic composition of this invention include antigens from an animal, a plant, a bacteria, a protozoan, a parasite, a virus or a combination thereof. The antigenic or immunogenic agent for use in the immunogenic composition of this invention may be any substance that under appropriate conditions results in an immune response in a subject, including, but not limited to, polypeptides, peptides, proteins, glycoproteins, and polysaccharides.

The immunogenic composition of this invention may comprise one or more antigenic or immunogenic agents. The amount of the antigenic or immunogenic agent used in the compositions of this invention may vary depending on the chemical nature and the potency of the antigenic or immunogenic agent. Typically, the starting concentration of the antigenic or immunogenic agent in the composition of this invention is the amount that is conventionally used for eliciting the desired immune response, using the conventional routes of administration, e.g., intramuscular injection. The concentration of the antigenic or immunogenic agent in the composition of this invention is then adjusted, e.g., by dilution using a diluent, so that an effective protective immune response is achieved as assessed using standard methods known in the art and described herein.

The antigenic or immunogenic agent may be any viral peptide, protein, polypeptide, or a fragment thereof derived from a virus including, but not limited to, RSV-viral proteins, e.g., RSV F glycoprotein, RSV G glycoprotein, influenza viral proteins, e.g., influenza virus neuramimidase, influenza virus hemagglutinin, herpes simplex viral protein, e.g., herpes simplex virus glycoprotein including for example, gB, gC, gD, and gE.

The antigenic or immunogenic agent for use in the composition of this invention may be an antigen of a pathogenic virus, including as examples and not by limitation: adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxyiridae (e.g., chordopoxyirinae, parapoxvirus, avipoxvirus, capripoxvirus, leporiipoxvirus, suipoxvirus, molluscipoxvirus, and entomopoxyirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), and metapneumovirus (e.g., avian pneumovirus and human metapneumovirus)), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

The antigenic or immunogenic agent used in the composition of this invention may be an infectious disease agent including, but not limited to, influenza virus hemagglutinin (Genbank accession no. J02132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), core protein, matrix protein or other protein of Dengue virus (Genbank accession no. M119197; Hahn et al., 1988, Virology 162:167-180), measles virus hemagglutinin (Genbank accession no. M81899; Rota et al., 1992, Virology 188:135-142), herpes simplex virus type 2 glycoprotein gB (Genbank accession no. M14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diptheria toxin (Audibert et al., 1981, Nature 289:543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gill (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin-neuramimidase, swine flu hemagglutinin, swine flu neuramimidase, foot and mouth disease virus, hog cholera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus (Gonzales-Scarano et al., 1982, Virology 120:42), neonatal calf diarrhea virus (Matsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, in Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), antigen of equine influenza virus or equine herpes virus (e.g., equine influenza virus type A/Alaska 91 neuraminidase, equine influenza virus type A/Miami 63 neuraminidase, equine influenza virus type A/Kentucky 81 neuraminidase equine herpes virus type 1 glycoprotein B, and equine herpes virus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53.

The antigenic or immunogenic agent in the composition of this invention may also be a cancer antigen or a tumor antigen. Any cancer or tumor antigen known to one skilled in the art may be used in accordance with the intradermal vaccine formulations of the invention including, but not limited to, KS 1/4 pan-carcinoma antigen (Perez and Walker, 1990, J. Immunol. 142:3662-3667; Bumal, 1988, Hybridoma 7(4):407-415), ovarian carcinoma antigen (CA125) (Yu et al., 1991, Cancer Res. 51(2):468-475), prostatic acid phosphate (Tailor et al., 1990, Nucl. Acids Res. 18(16):4928), prostate specific antigen (Henttu and Vihko, 1989, Biochem. Biophys. Res. Comm. 160(2):903-910; Israeli et al., 1993, Cancer Res. 53:227-230), melanoma-associated antigen p97 (Estin et al., 1989, J. Natl. Cancer Instit. 81(6):445-446), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-63; Mittelman et al., 1990, J. Clin. Invest. 86:2136-2144), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immunospecifically. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos. In one embodiment, the antigen is a Tcell receptor derived peptide from a Cutaneous Tcell Lymphoma (see, Edelson, 1998, The Cancer Journal 4:62).

The antigenic or immunogenic agent in the composition of this invention may comprise a virus, against which an immune response is desired. In certain cases, the composition of this invention comprise recombinant or chimeric viruses. In other cases, the immunogenic composition of this invention comprises a virus which is attenuated. Production of recombinant, chimeric and attenuated viruses may be performed using standard methods known to one skilled in the art. This invention also encompasses a live recombinant viral vaccine or an inactivated recombinant viral vaccine to be formulated in accordance with the invention. A live vaccine may be preferred because multiplication in the host leads to a prolonged stimulus of similar kind and magnitude to that occurring in natural infections, and therefore, confers substantial, long-lasting immunity. Production of such live recombinant virus vaccine formulations may be accomplished using conventional methods involving propagation of the virus in cell culture or in the allantois of the chick embryo followed by purification.

The recombinant virus may be non-pathogenic to the subject to which it is administered. In this regard, the use of genetically engineered viruses for vaccine purposes may require the presence of attenuation characteristics in these strains. The introduction of appropriate mutations (e.g., deletions) into the templates used for transfection may provide the novel viruses with attenuation characteristics. For example, specific missense mutations which are associated with temperature sensitivity or cold adaptation can be made into deletion mutations. These mutations should be more stable than the point mutations associated with cold or temperature sensitive mutants and reversion frequencies should be extremely low.

Alternatively, chimeric viruses with “suicide” characteristics may be constructed for use in the composition of this invention. Such viruses would go through only one or a few rounds of replication within the host. When used as a vaccine, the recombinant virus would go through limited replication cycle(s) and induce a sufficient level of immune response but it would not go further in the human host and cause disease.

Alternatively, inactivated (killed) virus may be formulated in accordance with the invention. Inactivated vaccine formulations may be prepared using conventional techniques to “kill” the chimeric viruses. Inactivated vaccines are “dead” in the sense that their infectivity has been destroyed. Ideally, the infectivity of the virus is destroyed without affecting its immunogenicity. In order to prepare inactivated vaccines, the chimeric virus may be grown in cell culture or in the allantois of the chick embryo, purified by zonal ultracentrifugation, inactivated by formaldehyde or β-propiolactone, and pooled.

Completely foreign epitopes, including antigens derived from other viral or non-viral pathogens can also be engineered into the virus for use in the composition of this invention. For example, antigens of non-related viruses such as HIV (gp160, gp120, gp41) parasite antigens (e.g., malaria), bacterial or fungal antigens or tumor antigens can be engineered into the attenuated strain.

Virtually any heterologous gene sequence may be constructed into the chimeric viruses for use in the composition of this invention. Preferably, heterologous gene sequences are moieties and peptides that act as biological response modifiers. Preferably, epitopes that induce a protective immune response to any of a variety of pathogens, or antigens that bind neutralizing antibodies may be expressed by or as part of the chimeric viruses. For example, heterologous gene sequences that can be constructed into the chimeric viruses include, but are not limited to, influenza and parainfluenza hemagglutinin neuraminidase and fusion glycoproteins such as the HN and F genes of human PIV3. In addition, in the case that cytokines are used with the gene-based antigenic or immunogenic agents, heterologous gene sequences that can be engineered into the chimeric viruses include those that encode such cytokines.

Other heterologous sequences may be derived from tumor antigens, and the resulting chimeric viruses be used to generate an immune response against the tumor cells leading to tumor regression in vivo. In accordance with the present invention, recombinant viruses may be engineered to express tumor-associated antigens (TAAs), including but not limited to, human tumor antigens recognized by T cells (Robbins and Kawakami, 1996, Curr. Opin. Immunol. 8:628-636, incorporated herein by reference in its entirety); melanocyte lineage proteins, including gp100, MART-1/MelanA, TRP-1 (gp75) and tyrosinase; tumor-specific widely shared antigens, such as MAGE-1, MAGE-3, BAGE, GAGE-1, GAGE-1, N-acetylglucosaminyltransferase-V and p15; tumor-specific mutated antigens, such as β-catenin, MUM-1 and CDK4; non-melanoma antigens for breast, ovarian, cervical and pancreatic carcinoma, HER-2/neu, human papillomavirus-E6, -E7, MUC-1.

The antigenic or immunogenic agent for use in the composition of this invention may include one or more of the select agents and toxins as identified by the Center for Disease Control. In certain cases, the select agent for use in the composition of this invention may comprise one or more antigens from Staphyloccocal enterotoxin B, Botulinum toxin, protective antigen for Anthrax, and Yersinia pestis. A non-limiting examples of select agents and toxins for use in the composition of this invention are listed in Table I:

TABLE I
SELECT AGENTS

HHS NON-OVERLAP SELECT AGENTS AND
TOXINS
Crimean-Congo haemorrhagic fever virus
Coccidioides posadasii
Ebola viruses
Cercopithecine herpes virus 1 (Herpes B virus)
Lassa fever virus
Marburg virus
Monkeypox virus
Rickettsia prowazekii
Rickettsia rickettsii
South American haemorrhagic fever viruses
Junin
Machupo
Sabia
Flexal
Guanarito
Tick-borne encephalitis complex (flavi) viruses
Central European tick-borne encephalitis
Far Eastern tick-borne encephalitis
Russian spring and summer encephalitis
Kyasanur forest disease
Omsk hemorrhagic fever
Variola major virus (Smallpox virus)
Variola minor virus (Alastrim)
Yersinia pestis
Abrin
Conotoxins
Diacetoxyscirpenol
Ricin
Saxitoxin
Shiga-like ribosome inactivating proteins
Tetrodotoxin
HIGH CONSEQUENCE LIVESTOCK
PATHOGENS AND TOXINS/SELECT AGENTS (OVERLAP
AGENTS)
Bacillus anthracis
Brucella abortus
Brucella melitensis
Brucella suis
Burkholderia mallei
(formerly Pseuodomonas mallei)
Burkholderia pseudomallei
(formerly Pseuodomonas pseudomallei)
Botulinum neurotoxin producing species of
Clostridium
Coccidioides immitis
Coxiella burnetii
Eastern equine encephalitis virus
Hendra virus
Francisella tularensis
Nipah Virus
Rift Valley fever virus
Venezuelan equine encephalitis virus
Botulinum neurotoxin
Clostridium perfringens epsilon toxin
Shigatoxin
Staphylococcal enterotoxin
T-2 toxin
USDA HIGH CONSEQUENCE
LIVESTOCK PATHOGENS AND TOXINS
(NON-OVERLAP AGENTS AND TOXINS
Akabane virus
African swine fever virus
African horse sickness virus
Avian influenza virus (highly
pathogenic)
Blue tongue virus (Exotic)
Bovine spongiform
encephalopathy agent
Camel pox virus
Classical swine fever virus
Cowdria ruminantium
(Heartwater)
Foot and mouth disease virus
Goat pox virus
Lumpy skin disease virus
Japanese encephalitis virus
Malignant catarrhal fever virus
(Exotic)
Menangle virus
Mycoplasma capricolumi
M.F38/M. mycoides capri
Mycoplasm mycoides mycoides
Newcastle disease virus (VVND)
Peste Des Petits Ruminants virus
Rinderpest virus
Sheep pox virus
Swine vesicular disease virus
Vesicular stomatitis virus
(Exotic)
LISTED PLANT PATHOGENS
Liberobacter africanus
Liberobacter asiaticus
Peronosclerospora
phillippinensis
Phakopsora pachyrhizi
Plum Pox Potyvirus
Ralstonia solanacearum race 3,
biovar 2
Schlerophthora rayssiae var zeae
Synchytrium endobioticum
Xanthomonas oryzae
Xylella fastidiosa (citrus
variegated chlorosis strain)

5.1.3 Influenza Virus Antigens

Exemplary immunogenic compositions of the invention for intradermal delivery are influenza virus vaccines, which may comprise one or more influenza virus antigens. Preferably, the influenza virus antigens used in the intradermal immunogenic formulations of the invention are surface antigens, including, but not limited to, haemagglutinin and neuraminidase antigens or a combination thereof. The influenza virus antigens may form part of a whole influenza vaccine formulations. Alternatively, the influenza virus antigens can be present as purified or substantially purified antigens. Techniques for isolating and purifying influenza virus antigens are known to one skilled in the art and are contemplated in the present invention. An example of a haemagglutinin/neuraminidase preparation suitable for use in the compositions of the present invention is the “Fluvirin” product manufactured and sold by Evans Medical Limited of Speke, Merseyside, United Kingdom; and see also S. Renfrey and A. Watts, 1994 Vaccine, 12(8): 747-752; which is incorporated herein by reference in its entirety.

The influenza vaccines useful in the intradermal immunogenic compositions of the present invention may be any commercially available influenza vaccine, preferably a trivalent subunit vaccine, e.g., FLUZONE™ attenuated flu vaccine (Aventis Pasteur, Inc. Swiftwater, Pa.). The influenza vaccine formulations used according to the invention have a therapeutic efficacy at a dose which is lower than the conventional dose used for intramuscular delivery of influenza vaccines. The influenza vaccine used as the intradermal immunogenic composition of the invention may be a non-live influenza antigenic preparation, preferably a split influenza or a subunit antigenic preparation, prepared using common methods known in the art. Most preferably, the influenza vaccine used in accordance with the invention is a trivalent vaccine.

The invention encompasses influenza vaccine formulations comprising a non-live influenza antigenic preparation, preferably a split influenza preparation or a subunit antigenic preparation prepared from a live virus. Most preferably the influenza antigenic preparation is a split influenza antigenic preparation.

The influenza vaccine formulation used in accordance with the present invention may contain influenza virus antigens from a single viral strain, or from a plurality of strains. For example, the influenza vaccine formulation may contain antigens taken from up to three or more viral strains. Purely by way of example, the influenza vaccine formulation may contain antigens from one or more strains of influenza A together with antigens from one or more strains of influenza B. Examples of influenza strains are strains of influenza A/Texas/36/91, A/Nanchang/933/95 and B/Harbin/7/94.

In a most preferred embodiment, the influenza vaccine formulation used in accordance with the invention comprises a commercially available influenza vaccine, FLUZONE™, which is an attenuated flu vaccine (Connaught Laboratories, Swiftwater, Pa.). FLUZONE is a trivalent subvirion vaccine comprising 15 ug/dose of each the HAs from influenza A/Texas/36/91 (NINI), A/Beijing/32/92 (H3N2) and B/Panama, 45/90 viruses.

Preferably, the influenza vaccine formulations used in accordance with the invention have a lower quantity of haemagglutinin than conventional vaccines and are administered in a lower volume. In some embodiments, the quantity of haemagglutinin per strain of influenza is about 1-7.5 μg, more preferably approximately 3 μg or approximately 5 μg, which is about one fifth or one third, respectively, of the dose of haemagglutinin used in conventional vaccines for intramuscular administration.

The volume of a dose of an influenza vaccine formulation according to the invention is between 0.025 ml and 2.5 ml, more preferably approximately 0.1 ml or approximately 0.2 ml. In a specific embodiment, the invention encompasses a 50 μl dose volume of the influenza vaccine. A 0.1 ml dose is approximately one fifth of the volume of a conventional intramuscular flu vaccine dose. The volume of liquid that can be administered intradermally depends, in part, upon the site of the injection. For example, for an injection in the deltoid region, 0.1 ml is the maximum preferred volume whereas in the lumbar region a larger volume e.g. about 0.2 ml can be given.

Standards are applied internationally to measure the efficacy of influenza vaccines. The European Union official criteria for an effective vaccine against influenza are set out in the table below. Theoretically, to meet the European Union requirements, and thus be approved for sale in the EU, an influenza vaccine has to meet one of the criteria in the table below, for all strains of influenza included in the vaccine. However in practice, at least two or more, probably all three of the criteria will need to be met for all strains, particularly for a new vaccine coming onto the market. Under some circumstances, two criteria may be sufficient. For example, it may be acceptable for two of the three criteria to be met by all strains while the third criterion is met by some but not all strains (e.g. two out of three strains). The requirements are different for adult populations (18-60 years) and elderly populations (>60 years).

TABLE II
EU STANDARDS FOR AN EFFECTIVE INFLUENZA VACCINE

| | 18-60 years | >60 years |
|---|---|---|
| Seroconversion rate | >40% | >30% |
| Conversion factor | >2.5 | >2.0 |
| Protection rate | >70% | >60% |

Seroconversion rate is defined as the percentage of recipients who have at least a 4-fold increase in serum haemagglutinin inhibition (HI) titres after vaccination, for each vaccine strain. Conversion factor is defined as the fold increase in serum HI geometric mean titres (C3MTs) after vaccination, for each vaccine strain. Protection rate is defined as the percentage of recipients with a serum HI titre equal to or greater than 1:40 after vaccination (for each vaccine strain) and is normally accepted as indicating protection.

The influenza vaccine formulations of the invention meet some or all of the EU criteria for influenza vaccines as set out hereinabove, such that the vaccine is approvable in Europe. Preferably, at least two out of the three EU criteria are met, for all strains of influenza represented in the vaccine. More preferably, at least two criteria are met for all strains and the third criterion is met by all strains or at least by all but one of the strains. More preferably, all strains present meet all three of the criteria. Preferably, the influenza vaccine formulations of the invention additionally meet some or all criteria of the Federal Drug Administration and/or USPHS requirements for the current influenza vaccines.

5.2 Preparation of the Dermal Compositions of the Invention

5.2.1 Preparation of the Intradermal Compositions

The intradermal immunogenic composition of this invention may be prepared by any method that results in a stable, sterile, injectable formulation. Preferably, the method for preparing an intradermal immunogenic composition of this invention comprises: providing a solution of the adjuvant; providing a solution of the antigenic or immunogenic agent; and combining the solution of the adjuvant and the solution of the antigenic or immunogenic agent to form the inoculum, e.g., the solution to be injected to the intradermal compartment.

In one embodiment, the adjuvant, e.g., in a particulate form, may be dissolved in a solution of the antigenic or immunogenic agent, such that a stable, sterile, injectable formulation is formed. Alternatively, the antigenic or immunogenic agent may be particulate and dissolved in the adjuvant solution such that a stable, sterile, injectable formulation is formed. For enhanced performance of the immunogenic composition of this invention, the antigenic or immunogenic agent should be uniformly dispersed throughout the composition.

In another specific embodiment, the adjuvant and the antigenic or immunogenic agent are mixed prior to administration to a subject. Alternatively, the adjuvant and the antigenic or immunogenic agent can be mixed during administration in an intradermal delivery device.

The amount of the antigenic or immunogenic agent used in the immunogenic composition of this invention may vary depending on the chemical nature and the potency of the antigenic or immunogenic agent and the specific adjuvant used. Typically, the starting concentration of the antigenic or immunogenic agent in the composition of this invention is the amount that is conventionally used for eliciting the desired immune response, using the conventional routes of administration, e.g., intramuscular injection. The concentration of the antigenic or immunogenic agent is then adjusted, e.g., by dilution using a diluent, in the dermal immunogenic compositions of the invention so that an effective protective immune response is achieved as assessed using standard methods known in the art and described herein.

The amount of the adjuvant used in the immunogenic composition of this invention may vary depending on the chemical nature of the adjuvant and the specific antigenic or immunogenic agent used. Certain preferred concentrations of the adjuvant compounds, described in Section 5.1.1, above, can generally be used effectively with many antigenic or immunogenic agent. One of ordinary skill in the art would appreciate, however, that depending on the individual adjuvant and the antigenic or immunogenic agent, the amount of adjuvant may be adjusted using the methods that are substantially identical to those disclosed above for the determination of an effective amount of the antigenic or immunogenic agent, as well as other methods conventionally known in the art.

5.2.2 Preparation of Epidermal Compositions

The epidermal immunogenic compositions of the invention may be prepared by any method that results in a stable, sterile formulation such as those known in the art and disclosed in Patent Application Publication Nos. US 2003/0191085 and US 2003/0093040, both of which are hereby incorporated by reference in their entirety. They can be delivered, inter alia, in the form of dry powders, gels, solutions, suspensions, and creams.

The epidermal immunogenic compositions may be delivered into the epidermal compartment of skin in any pharmaceutically acceptable form. In one embodiment the epidermal immunogenic compositions is applied to the skin and an abrading device is then moved or rubbed reciprocally over the skin and the substance. It is preferred that the minimum amount of abrasion to produce the desired result be used. Determination of the appropriate amount of abrasion for a selected epidermal immunogenic compositions is within the ordinary skill in the art. In another embodiment the epidermal immunogenic composition may be applied in dry form to the abrading surface of the delivery device prior to application. In this embodiment, a reconstituting liquid is applied to the skin at the delivery site and the formulation-coated abrading device is applied to the skin at the site of the reconstituting liquid. It is then moved or rubbed reciprocally over the skin so that the vaccine formulation becomes dissolved in the reconstituting liquid on the surface of the skin and is delivered simultaneously with abrasion. Alternatively, a reconstituting liquid may be contained in the abrading device and released to dissolve the vaccine formulation as the device is applied to the skin for abrasion. It has been found that certain epidermal immunogenic compositions, may also be coated on the abrading device in the form of a gel.

5.3 Administration of the Compositions of the Invention

5.3.1 Intradermal Administration

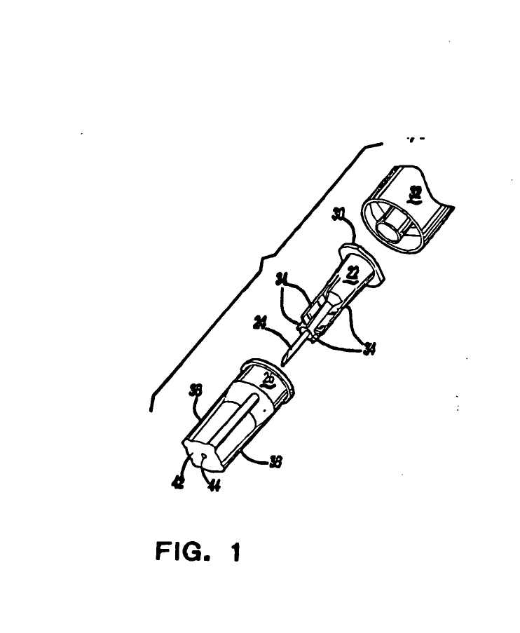

The present invention encompasses methods for intradermal delivery of the immunogenic compositions such as vaccine formulations described and exemplified herein to the intradermal compartment of a subject's skin, preferably by directly and selectively targeting the intradermal space. Once the intradermal immunogenic composition is prepared in accordance to the methods described supra, the inoculum is typically transferred to an injection device for intradermal delivery, e.g., a syringe. Preferably, the inoculum is administered to the intradermal compartment of a subject's skin within 1 hour of preparation. The intradermal immunogenic compositions of the invention are administered using any of the intradermal devices and methods disclosed in U.S. Pat. No. 6,494,865; Patent Application Publication Nos. US 2005/0096632, US 2002/0095134, US 2002/0156453, and US 2003/0100885; or International Publication No.'s EP 10922 444, published Apr. 18, 2001; WO 01/02178, published Jan. 10, 2002; and WO 02/02179, published Jan. 10, 2002; all of which are incorporated herein by reference in their entirety. Exemplary devices are shown in FIGS. 1-3.

The present invention improves the clinical utility and therapeutic efficacy of immunogenic compositions including vaccine formulations described herein by specifically and selectively, preferably directly, targeting the intradermal space. The intradermal immunogenic compositions may be delivered to the intradermal space as a bolus or by infusion.

The administration site can be defined as the area immediately surrounding the needle penetration site. Blebbs can expand outward from a needle penetration site by 0 to 10 mm. Transient blebbs can reach 2 cm in diameter with larger injection volumes. The inventors have discovered unexpectedly that the delivery of the immunogenic compositions described and exemplified herein to the dermis provides for efficacious and/or improved responsiveness to the immunogenic compositions. The immunogenic compositions of the invention as administered to the intradermal compartment have an improved adsorption and/or cellular uptake within the intradermal space. The immunological response to a immunogenic compositions delivered according to the methods of the invention has been found to be equivalent to or improved over conventional routes of delivery, e.g., intramuscular.

The present invention provides a method to improve the availability of a immunogenic compositions of the invention to the immune cells residing in the skin, e.g., antigen presenting cells, in order to effectuate an antigen-specific immune response to the immunogenic compositions by accurately targeting the intradermal space. Preferably, the methods of the invention, allow for smaller doses of the intradermal immunogenic compositions to be administered via the intradermal route.

The intradermal methods of administration comprise microneedle-based injection and infusion systems or any other means to accurately target the intradermal space. The intradermal methods of administration encompass not only microdevice-based injection means, but other delivery methods such as needless or needle-free ballistic injection of fluids or powders into the intradermal space, Mantoux-type intradermal injection, enhanced iontophoresis through microdevices, and direct deposition of fluid, solids, or other dosing forms into the skin.

In a specific embodiment, the intradermal immunogenic compositions of the invention are administered to an intradermal compartment of a subject's skin using an intradermal Mantoux type injection, see, e.g., Flynn et al., 1994, Chest 106: 1463-5, which is incorporated herein by reference in its entirety.

In another specific embodiment, the immunogenic composition of the invention is delivered to the intradermal compartment of a subject's skin using the following exemplary method. The intradermal immunogenic composition as prepared in accordance to methods disclosed herein is drawn up into a syringe, e.g., a 1 mL latex free syringe with a 20 gauge needle; after the syringe is loaded it is replaced with a 30 gauge needle for intradermal administration. The skin of the subject, e.g., mouse, is approached at the most shallow possible angle with the bevel of the needle pointing upwards, and the skin pulled tight. The injection volume is then pushed in slowly over 5-10 seconds forming the typical “bleb” and the needle is subsequently slowly removed. Preferably, only one injection site is used. More preferably, the injection volume is no more than 100 μL, due in part, to the fact that a larger injection volume may increase the spill over into the surrounding tissue space, e.g., the subcutaneous space.

The invention encompasses the use of conventional injection needles, catheters or microneedles of all known types, employed singularly or in multiple needle arrays. The terms “needle” and “needles” as used herein are intended to encompass all such needle-like structures. The term “microneedles” as used herein are intended to encompass structures smaller than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes.

The intradermal delivery of the immunogenic compositions of the invention may use ballistic fluid injection devices, powder jet delivery devices, piezoelectric, electromotive, electromagnetic assisted delivery devices, gas-assisted delivery devices, which directly penetrate the skin to directly deliver the immunogenic compositions of the invention to the targeted location within the dermal space.

The actual method by which the intradermal immunogenic compositions of the invention are targeted to the intradermal space is not critical as long as it penetrates the skin of a subject to the desired targeted depth within the intradermal space without passing through it. In most cases, skin is penetrated to a depth of about 0.5-2 mm. Regardless of the specific intradermal device and method of delivery, the intradermal immunogenic compositions is preferably deposit to a depth of at least 0.3 mm, more preferably at least 0.5 mm up to a depth of no more than 2.5 mm, more preferably no more than 2.0 mm, and most preferably no more than 1.7 mm. The methods of the invention comprise use of delivery devices as disclosed infra which place the needle outlet at an appropriate depth in the intradermal space and control the volume and rate of fluid delivery provide accurate delivery of the formulation to the desired location without leakage.

The invention encompasses use of devices comprising microneedles which have a length sufficient to penetrate the intradermal space (the “penetration depth”) and an outlet at a depth within the intradermal space (the “outlet depth”) which allows the skin to seal around the needle against the backpressure which tends to force the delivered formulation toward the skin surface. In general, the needle is no more than about 2 mm long, preferably about 300 μm to 2 mm long, most preferably about 500 μm to 1 mm long. The needle outlet is typically at a depth of about 250 μm to 2 mm when the needle is inserted in the skin, preferably at a depth of about 750 μm to 1.5 mm, and most preferably at a depth of about 1 mm. The exposed height of the needle outlet and the depth of the outlet within the intradermal space influence the extent of sealing by the skin around the needle. That is, at a greater depth a needle outlet with a greater exposed height will still seal efficiently whereas an outlet with the same exposed height will not seal efficiently when placed at a shallower depth within the intradermal space. Typically, the exposed height of the needle outlet will be from 0 to about 1 mm, preferably from 0 to about 300 μm. A needle outlet with an exposed height of 0 has no bevel and is at the tip of the needle. in this case, the depth of the outlet is the same as the depth of penetration of the needle. A needle outlet which is either formed by a bevel or by an opening through the side of the needle has a measurable exposed height.

In some embodiments, the immunogenic compositions are delivered at a targeted depth just under the stratum corneum and encompassing the epidermis and upper dermis, e.g., about 0.025 mm to about 2.5 mm. In order to target specific cells in the skin, the preferred target depth depends on the particular cell being targeted and the thickness of the skin of the particular subject. For example, to target the Langerhan's cells in the dermal space of human skin, delivery would need to encompass, at least, in part, the epidermal tissue depth typically ranging from about 0.025 mm to about 0.2 mm in humans.

In some embodiments, when the vaccine formulations require systemic circulation, the preferred target depth would be between, at least about 0.4 mm and most preferably, at least about 0.5 mm, up to a depth of no more than about 2.5 mm, more preferably, no more than about 2.0 mm and most preferably, no more than about 1.7 mm. Targeting the vaccine formulations predominately at greater depths and/or into a lower portion of the reticular dermis is usually considered to be less desirable.

The invention provides a method for an improved method of delivering the immunogenic compositions including the vaccines formulations into the intradermal compartment of a subject's skin comprising the steps of providing a drug delivery device, e.g., such as those exemplified in FIGS. 1-3, including a needle cannula having a forward needle tip and the needle cannula being in fluid communication with a formulation contained in the drug delivery device and including a limiter portion surrounding the needle cannula and the limiter portion including a skin engaging surface, with the needle tip of the needle cannula extending from the limiter portion beyond the skin engaging surface a distance equal to approximately 0.5 mm to approximately 3.0 mm and the needle cannula having a fixed angle of orientation relative to a plane of the skin engaging surface of the limiter portion, inserting the needle tip into the skin of an animal and engaging the surface of the skin with the skin engaging surface of the limiter portion, such that the skin engaging surface of the limiter portion limits penetration of the needle cannula tip into the dermis layer of the skin of the animal, and expelling the formulation from the drug delivery device through the needle cannula tip into the skin of the subject.

Also, in other preferred embodiments, the invention encompass selecting an injection site on the skin of the subject, cleaning the injection site on the skin of the subject prior to expelling the immunogenic compositions of the invention from the drug delivery device into the skin of the subject. In addition, the method comprises filling the drug delivery device with the immunogenic compositions of the invention. Further, the method comprises pressing the skin engaging surface of the limiter portion against the skin of the subject and applying pressure, thereby stretching the skin of the subject, and withdrawing the needle cannula from the skin after injecting the immunogenic compositions. Still further, the step of inserting the forward tip into the skin is further defined by inserting the forward tip into the skin to a depth of from approximately 1.0 mm to approximately 2.0 mm, and most preferably into the skin to a depth of 1.5 mm±0.2 to 0.3 mm. FIGS. 1-3 exemplify specific embodiments of the intradermal methods of the invention.

In the preferred embodiment of the method, the step of inserting the forward tip into the skin of the subject is further defined by inserting the forward tip into the skin at an angle being generally perpendicular to the skin within about fifteen degrees, with the angle most preferably being generally ninety degrees to the skin, within about five degrees, and the fixed angle of orientation relative to the skin engaging surface is further defined as being generally perpendicular. In the preferred embodiment, the limiter surrounds the needle cannula, having a generally planar flat skin engaging surface. Also, the drug delivery device comprises a syringe having a barrel and a plunger received within the barrel and the plunger being depressible to expel the substance from the delivery device through the forward tip of the needle cannula, e.g., see FIGS. 1-3.

In a preferred embodiment, expelling the immunogenic composition from the delivery device is further defined by grasping the hypodermic needle with a first hand and depressing the plunger with an index finger of a second hand and expelling the immunogenic composition from the delivery device by grasping the hypodermic needle with a first hand and depressing the plunger on the hypodermic needle with a thumb of a second hand, with the step of inserting the forward tip into the skin of the animal further defined by pressing the skin of the animal with the limiter. In addition, the method may further comprise the step of attaching a needle assembly to a tip of the barrel of the syringe with the needle assembly including the needle cannula and the limiter, and may comprise the step of exposing the tip of the barrel before attaching the needle assembly thereto by removing a cap from the tip of the barrel. Alternatively, the step of inserting the forward tip of the needle into the skin of the subject may be further defined by simultaneously grasping the hypodermic needle with a first hand and pressing the limiter against the skin of the animal thereby stretching the skin of the animal, and expelling the substance by depressing the plunger with an index finger of the first hand or expelling the substance by depressing the plunger with a thumb of the first hand. The method further encompasses withdrawing the forward tip of the needle cannula from the skin of the subject after the substance has been injected into the skin of the subject. Still further, the method encompasses inserting the forward tip into the skin preferably to a depth of from approximately 1.0 mm to approximately 2.0 mm, and most preferably to a depth of 1.5 mm±0.2 to 0.3 mm.

Preferably, prior to inserting the needle cannula 24 (see FIGS. 1-3), an injection site upon the skin of the subject is selected and cleaned. Subsequent to selecting and cleaning the site, the forward end 40 of the needle cannula 24 is inserted into the skin of the subject at an angle of generally 90 degrees until the skin engaging surface 42 contacts the skin. The skin engaging surface 42 prevents the needle cannula 42 from passing through the dermis layer of the skin and injecting the immunogenic composition into the subcutaneous layer. While the needle cannula 42 is inserted into the skin, the vaccine formulation is intradermally injected. The vaccine formulation may be prefilled into the syringe 60, either substantially before and stored therein just prior to making the injection. Several variations of the method of performing the injection may be utilized depending upon individual preferences and syringe type. In any event, the penetration of the needle cannula 42 is most preferably no more than about 1.5 mm because the skin engaging surface 42 prevents any further penetration.

Also, during the administration of an intradermal injection, the forward end 40 of the needle cannula 42 is embedded in the dermis layer of the skin which results in a reasonable amount of back pressure during the injection of the vaccine formulation of the invention. This back pressure could be on the order of 76 psi. In order to reach this pressure with a minimal amount of force having to be applied by the user to the plunger rod 66 of the syringe, a syringe barrel 60 with a small inside diameter is preferred such as 0.183″ (4.65 mm) or less. The method of this invention thus comprises selecting a syringe for injection having an inside diameter of sufficient width to generate a force sufficient to overcome the back pressure of the dermis layer when the vaccine formulation is expelled from the syringe to make the injection.

In addition, since intradermal injections are typically carried out with small volumes of the immunogenic composition to be injected, i.e., on the order of no more than 0.5 ml, and preferably around 0.1 ml, a syringe barrel 60 with a small inside diameter is preferred to minimize dead space which could result in wasted substance captured between the stopper 70 and the shoulder of the syringe after the injection is completed. Also, because of the small volumes of immunogenic composition, on the order of 0.1 ml, a syringe barrel with a small inside diameter is preferred to minimize air head space between the level of the substance and the stopper 70 during process of inserting the stopper. Further, the small inside diameter enhances the ability to inspect and visualize the volume of the immunogenic composition within the barrel of the syringe.

The intradermal administration methods useful for carrying out the invention include both bolus and infusion delivery of the immunogenic compositions to a subject, preferably a mammal, most preferably a human. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes.

The intradermal delivery of the formulations into the intradermal space may occur either passively, without application of the external pressure or other driving means to the vaccine formulations to be delivered, and/or actively, with the application of pressure or other driving means. Examples of preferred pressure generating means include pumps, syringes, elastomer membranes, gas pressure, piezoelectric, electromotive, electromagnetic pumping, or Belleville springs or washers or combinations thereof. If desired, the rate of delivery of the intradermal vaccine formulations of the invention may be variably controlled by the pressure-generating means.

The immunogenic compositions delivered or administered in accordance with the invention include solutions thereof in pharmaceutically acceptable diluents or solvents, suspensions, gels, particulates such as micro- and nanoparticles either suspended or dispersed, as well as in-situ forming vehicles of same.

The invention also encompasses varying the targeted depth of delivery of intradermal immunogenic compositions of the invention. The targeted depth of delivery of intradermal immunogenic compositions may be controlled manually by the practitioner, or with or without the assistance of an indicator to indicate when the desired depth is reached. Preferably however, the devices used in accordance with the invention have structural means for controlling skin penetration to the desired depth within the intradermal space. The targeted depth of delivery may be varied using any of the methods described in U.S. Pat. No. 6,494,865; Patent Application Publication Nos. US 2005/0096632, US 2002/0095134, US 2002/0156453, and US 2003/0100885; or International Publication No.'s EP 10922 444, published Apr. 18, 2001; WO 01/02178, published Jan. 10, 2002; and WO 02/02179, published Jan. 10, 2002; all of which are incorporated herein by reference in their entirety.

The dosage of the immunogenic compositions of the invention depends on the antigenic or immunogenic agent in the formulation. The dosage of the intradermal immunogenic compositions may be determined using standard immunological methods known in the art, for example, by first identifying doses effective to elicit a prophylactic or therapeutic immune response, e.g., by measuring the serum titer of antigen specific immunoglobulins, relative to a control formulation, e.g., a formulation simply consisting of the antigenic or immunogenic agent without a molecule as disclosed herein. Preferably, the effective dose is determined in an animal model, prior to use in humans. Most preferably, the optimal dose is determined in an animal whose skin thickness approximates closely to that of human skin, e.g., pig.

Intradermal vaccine formulations of the invention may also be administered on a dosage schedule, for example, an initial administration of the vaccine formulation with subsequent booster administrations. In particular embodiments, a second dose of the vaccine formulation is administered anywhere from two weeks to one year, preferably from one to six months, after the initial administration. Additionally, a third dose may be administered after the second dose and from three months to two years, or even longer, preferably 4 to 6 months, or 6 months to one year after the initial administration. In most preferred embodiments, however no booster immunization is required.

The immunogenic compositions of the invention are administered using any of the devices and methods known in the art or disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety. Preferably the devices for intradermal administration in accordance with the methods of the invention have structural means for controlling skin penetration to the desired depth within the intradermal space. This is most typically accomplished by means of a widened area or hub associated with the shaft of the dermal-access means that may take the form of a backing structure or platform to which the needles are attached. The length of microneedles as dermal-access means are easily varied during the fabrication process and are routinely produced in less than 2 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. The needle may eject its substance from the end, the side or both. Microneedles may be incorporated into a variety of devices such as holders and housings that may also serve to limit the depth of penetration. The dermal-access means of the invention may also incorporate reservoirs to contain the substance prior to delivery or pumps or other means for delivering the drug or other substance under pressure. Alternatively, the device housing the dermal-access means may be linked externally to such additional components.

The intradermal methods of administration comprise microneedle-based injection and infusion systems or any other means to accurately target the intradermal space. The intradermal methods of administration encompass not only microdevice-based injection means, but other delivery methods such as needle-less or needle-free ballistic injection of fluids or powders into the intradermal space, Mantoux-type intradermal injection, enhanced ionotophoresis through microdevices, and direct deposition of fluid, solids, or other dosing forms into the skin.

In some embodiments, the present invention provides a drug delivery device including a needle assembly for use in making intradermal injections. The needle assembly has an adapter that is attachable to prefillable containers such as syringes and the like. The needle assembly is supported by the adapter and has a hollow body with a forward end extending away from the adapter. A limiter surrounds the needle and extends away from the adapter toward the forward end of the needle. The limiter has a skin engaging surface that is adapted to be received against the skin of an animal such as a human. The needle forward end extends away from the skin engaging surface a selected distance such that the limiter limits the amount or depth that the needle is able to penetrate through the skin of an animal

In a specific embodiment, the hypodermic needle assembly for use in the methods of the invention comprises the elements necessary to perform the present invention directed to an improved method for delivering vaccine formulations into the skin of a subject's skin, preferably a human subject's skin, comprising the steps of providing a drug delivery device including a needle cannula having a forward needle tip and the needle cannula being in fluid communication with a substance contained in the drug delivery device and including a limiter portion surrounding the needle cannula and the limiter portion including a skin engaging surface, with the needle tip of the needle cannula extending from the limiter portion beyond the skin engaging surface a distance equal to approximately 0.5 mm to approximately 3.0 mm and the needle cannula having a fixed angle of orientation relative to a plane of the skin engaging surface of the limiter portion, inserting the needle tip into the skin of an animal and engaging the surface of the skin with the skin engaging surface of the limiter portion, such that the skin engaging surface of the limiter portion limits penetration of the needle cannula tip into the dermis layer of the skin of the animal, and expelling the substance from the drug delivery device through the needle cannula tip into the skin of the animal.

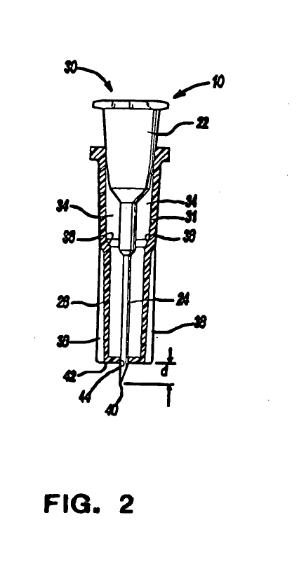

In a specific embodiment, the invention encompasses a drug delivery device as disclosed in FIGS. 1-3. FIGS. 1-3 illustrate an example of a drug delivery device which can be used to practice the methods of the present invention for making intradermal injections. The device 10 illustrated in FIGS. 1-3 includes a needle assembly 20 which can be attached to a syringe barrel 60. Other forms of delivery devices may be used including pens of the types disclosed in U.S. Pat. Nos. 5,279,586 and 6,248,095, and PCT Application No. WO 00/09135, the disclosure of which are hereby incorporated by reference in its entirety. The needle assembly 20 includes a hub 22 that supports a needle cannula 24. The limiter 26 receives at least a portion of the hub 22 so that the limiter 26 generally surrounds the needle cannula 24 as best seen in FIG. 2.

One end 30 of the hub 22 is able to be secured to a receiver 32 of a syringe. A variety of syringe types for containing the substance to be intradermally delivered according to the present invention can be used with a needle assembly designed, with several examples being given below. The opposite end of the hub 22 preferably includes extensions 34 that are nestingly received against abutment surfaces 36 within the limiter 26. A plurality of ribs 38 preferably are provided on the limiter 26 to provide structural integrity and to facilitate handling the needle assembly 20.

By appropriately designing the size of the components, a distance “d” between a forward end or tip 40 of the needle 24 and a skin engaging surface 42 on the limiter 26 can be tightly controlled. The distance “d” preferably is in a range from approximately 0.5 mm to approximately 3.0 mm, and most preferably around 1.5 mm±0.2 mm to 0.3 mm. When the forward end 40 of the needle cannula 24 extends beyond the skin engaging surface 42 a distance within that range, an intradermal injection is ensured because the needle is unable to penetrate any further than the typical dermis layer of an animal. Typically, the outer skin layer, epidermis, has a thickness between 50-200 microns, and the dermis, the inner and thicker layer of the skin, has a thickness between 1.5-3.5 mm. Below the dermis layer is subcutaneous tissue (also sometimes referred to as the hypodermis layer) and muscle tissue, in that order.

As can be best seen in FIG. 2, the limiter 26 includes an opening 44 through which the forward end 40 of the needle cannula 24 protrudes. The dimensional relationship between the opening 44 and the forward end 40 can be controlled depending on the requirements of a particular situation. In the illustrated embodiment, the skin engaging surface 42 is generally planar or flat and continuous to provide a stable placement of the needle assembly 20 against an animal's skin. Although not specifically illustrated, it may be advantageous to have the generally planar skin engaging surface 42 include either raised portions in the form of ribs or recessed portions in the form of grooves in order to enhance stability or facilitate attachment of a needle shield to the needle tip 40. Additionally, the ribs 38 along the sides of the limiter 26 may be extended beyond the plane of the skin engaging surface 42.

Regardless of the shape or contour of the skin engaging surface 42, the preferred embodiment includes enough generally planar or flat surface area that contacts the skin to facilitate stabilizing the injector relative to the subject's skin. In the most preferred arrangement, the skin engaging surface 42 facilitates maintaining the injector in a generally perpendicular orientation relative to the skin surface and facilitates the application of pressure against the skin during injection. Thus, in the preferred embodiment, the limiter has dimension or outside diameter of at least 5 mm. The major dimension will depend upon the application and packaging limitations, but a convenient diameter is less than 15 mm or more preferably 11-12 mm.

It is important to note that although FIGS. 1 and 2 illustrate a two-piece assembly where the hub 22 is made separate from the limiter 26, a device for use in connection with the invention is not limited to such an arrangement. Forming the hub 22 and limiter 26 integrally from a single piece of plastic material is an alternative to the example shown in FIGS. 1-2. Additionally, it is possible to adhesively or otherwise secure the hub 22 to the limiter 26 in the position illustrated in FIG. 1 so that the needle assembly 20 becomes a single piece unit upon assembly.

Having a hub 22 and limiter 26 provides the advantage of making an intradermal needle practical to manufacture. The preferred needle size is a small Gauge hypodermic needle, commonly known as a 30 Gauge or 31 Gauge needle. Having such a small diameter needle presents a challenge to make a needle short enough to prevent undue penetration beyond the dermis layer of an animal. The limiter 26 and the hub 22 facilitate utilizing a needle 24 that has an overall length that is much greater than the effective length of the needle, which penetrates the individual's tissue during an injection. With a needle assembly designed in accordance herewith, manufacturing is enhanced because larger length needles can be handled during the manufacturing and assembly processes while still obtaining the advantages of having a short needle for purposes of completing an intradermal injection.

FIG. 2 illustrates the needle assembly 20 secured to a drug container such as a syringe 60 to form the device 10. A generally cylindrical syringe body 62 can be made of plastic or glass as is known in the art. The syringe body 62 provides a reservoir 64 for containing the substance to be administered during an injection. A plunger rod 66 has a manual activation flange 68 at one end with a stopper 70 at an opposite end as known in the art. Manual movement of the plunger rod 66 through the reservoir 64 forces the substance within the reservoir 64 to be expelled out of the end 40 of the needle as desired.

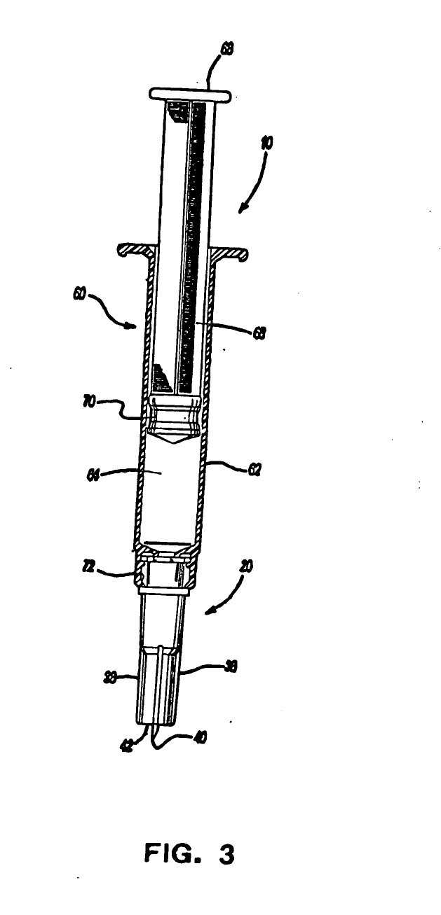

The hub 22 can be secured to the syringe body 62 in a variety of known manners. In one example, an interference fit is provided between the interior of the hub 22 and the exterior of the outlet port portion 72 of the syringe body 62. In another example, a conventional Luer fit arrangement is provided to secure the hub 22 on the end of the syringe 60. As can be appreciated from FIG. 3, such needle assembly designed is readily adaptable to a wide variety of conventional syringe styles.

This invention provides an intradermal needle injector that is adaptable to be used with a variety of syringe types. Therefore, this invention provides the significant advantage of facilitating manufacture and assembly of intradermal needles on a mass production scale in an economical fashion.

5.3.2 Epidermal Administration

The epidermal methods of administration comprise any method and device known in the art for accurately targeting the epidermal compartment such as those disclosed in Patent Application Publication Nos. US 2003/0191085 and US 2003/0093040, both of which are hereby incorporated by reference in their entirety. The present invention encompasses microabrading devices for accurately targeting the epidermal space. These devices may have solid or hollow micro-protrusions. The micro-protrusions can have a length up to about 500 microns. Suitable micro-protrusions have a length of about 50 to 500 microns. Preferably the microprotrusions have a length of about 50 to 300 microns and more preferably in the range of about 150 to 250 microns, with 180 to 220 microns being most preferred.

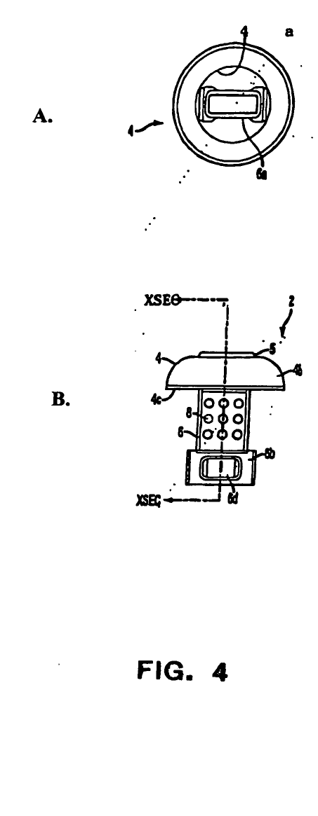

The microabrader devices that may be used in the methods of the invention are preferably a device capable of abrading the skin such as those exemplified in FIGS. 4-9. In preferred embodiments, the device is capable of abrading the skin thereby penetrating the stratum corneum without piercing the stratum corneum.

As used herein, “penetrating” refers to entering the stratum corneum without passing completely through the stratum corneum and entering into the adjacent layers. This is not to say that that the stratum corneum can not be completely penetrated to reveal the interface of the underlying layer of the skin. Piercing, on the other hand, refers to passing through the stratum corneum completely and entering into the adjacent layers below the stratum corneum. As used herein, the term “abrade” refers to removing at least a portion of the stratum corneum to increase the permeability of the skin without causing excessive skin irritation or compromising the skin's barrier to infectious agents. The term “abrasion” as used herein refers to disruption of the outer layers of the skin, for example by scraping or rubbing, resulting in an area of disrupted stratum corneum. This is in contrast to “puncturing” which produces discrete holes through the stratum corneum with areas of undisrupted stratum corneum between the holes.

Preferably, the devices used for epidermal delivery in accordance with the methods of the invention penetrate, but do not pierce, the stratum corneum. The immunogenic composition to be administered using the methods of this invention may be applied to the skin prior to abrading, simultaneous with abrading, or post-abrading.

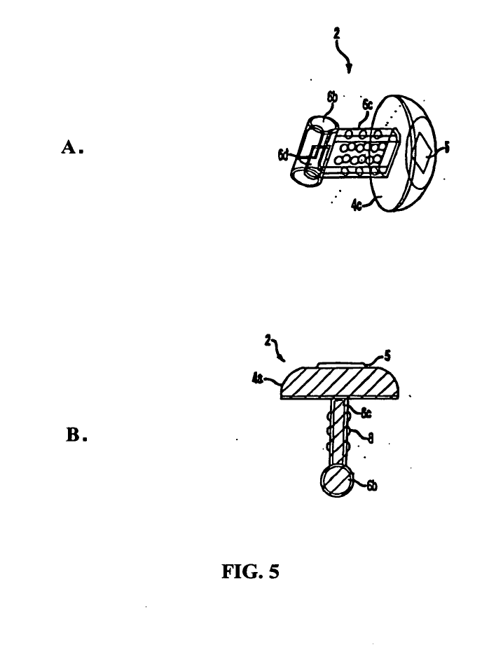

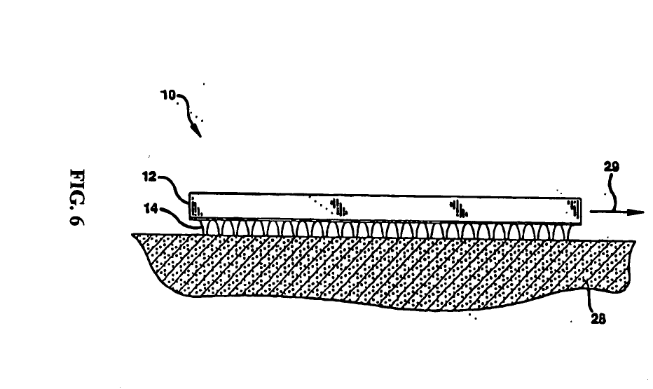

In a specific embodiment the invention encompasses a method for delivering an immunogenic composition into the skin of a patient comprising the steps of coating a patient's outer skin layer or a microabrader 2, see FIG. 4B with the formulation and moving microabrader 2 across the patient's skin to provide abrasions leaving furrows sufficient to permit entry of the formulation into the patient's viable epidermis. Due to the structural design of microabrader 2, the leading edge of microabrader 2 first stretches the patient's skin and then the top surface of microabrader 2 abrades the outer protective formulation e to enter the patient. After the initial abrasion of the outer protective skin layer, the trailing and leading edges of microabrader 2 can rub the surface of the abraded area working the formulation into the abraded skin area thereby improving its medicinal effect. As shown in FIGS. 4B, 5A and 5B, microabrader 2 includes base 4 onto which an abrading surface 5 can be mounted. Alternatively, the abrading surface may be integral with the base and fabricated as a single two-component part. Preferably, base 4 is a solid molded piece. In one embodiment, base 4 is configured with a mushroom-like crown 4b that curves upward and is truncated at the top. The top of base 4 is generally flat with abrading surface 5 being mounted thereon or integral therewith. Alternatively, the truncated top may have a recess for receiving abrading surface 5. In all embodiments, abrading surface 5 includes a platform with an array of microprotrusions that extends above the truncated top. In another embodiment of the microabrader, the handle, base and abrading surface may be integral with one another and fabricated as a single three-component device. Microabrader 2 is applied to a subject by moving microabrader 2 across the subject's skin with enough pressure to enable abrading surface 5 to open the outer protective skin or stratum corneum of the subject. The inward pressure applied to the base causes microabrader 2 to be pressed into the subject's skin. Accordingly, it is preferable that the height of the sloping mushroom-like crown 4b be sufficient to prevent the applied substance from flowing over and onto the facet 4c when microabrader 2 is being used. As will be described below, abrading surface 5 comprises an array of microprotrusions.

A handle 6 is attached to base 4 or may be integral with base 4. As shown in FIG. 5A, an upper end 6a of the handle may be either snap fit or friction fit between the inner circumferential sidewall 4a of base 4. Alternatively, as shown in FIGS. 4A and 5A, handle 6 may be glued (e.g., with epoxy) to the underside 4c of base 4. Alternatively, the handle and base may be fabricated (e.g., injection-molded) together as a single two-component part. The handle may be of a diameter that is less than the diameter of the base or may be of a similar diameter as the base. Underside 4c of base 4 may be flush with mushroom-like crown 4b or extend beyond the mushroom-like crown. The lower end 6b of handle 6 may be wider than the shaft 6c of handle 6 or may be of a similar diameter as shaft. Lower end 6b may include an impression 6d that serves as a thumb rest for a person administering the substance and moving microabrader 2. In addition, protrusions 8 are formed on the outside of handle 6 to assist a user in firmly gripping handle 6 when moving the same against or across a patient's skin.

As shown in the cross-section of FIG. 4B in FIG. 5B, lower end 6b may be cylindrical. Microabrader 2 may be made of a transparent material, as shown in FIG. 5A. Impressions 6d are disposed on both sides of the cylindrical lower end 6b to assist a person using microabrader 2 to grip the same. That is, the movement of microabrader 2 can be provided by hand or fingers. The handle 6, as well as the base 4, of the microabrader is preferably molded out of plastic or the like material. The microabrader 2 is preferably inexpensively manufactured so that the entire microabrader and abrading surface can be disposed after its use on one patient.

Abrading surface 5 is designed so that when microabrader 2 is moved across a patient's skin, the resultant abrasions penetrate the stratum corneum. Abrading surface 5 may be coated with a formulation desired to be delivered to the patient's viable epidermis.

In order to achieve the desired abrasions, the microabrader 2 should be moved across a patient's skin at least once. The patient's skin may be abraded in alternating directions. The structural design of the microabrader according to the invention enables the formulation to be absorbed more effectively thereby allowing less of the formulation to be applied to a patient's skin or coating abrading surface 5. Abrading surface 5 may be coated with a formulation desired to be delivered to the patient. In one embodiment, the formulation may be a powder disposed on abrading surface 5. In another embodiment, the formulation to be delivered may be applied directly to the patient's skin prior to the application and movement of microabrader 2 on the patient's skin.

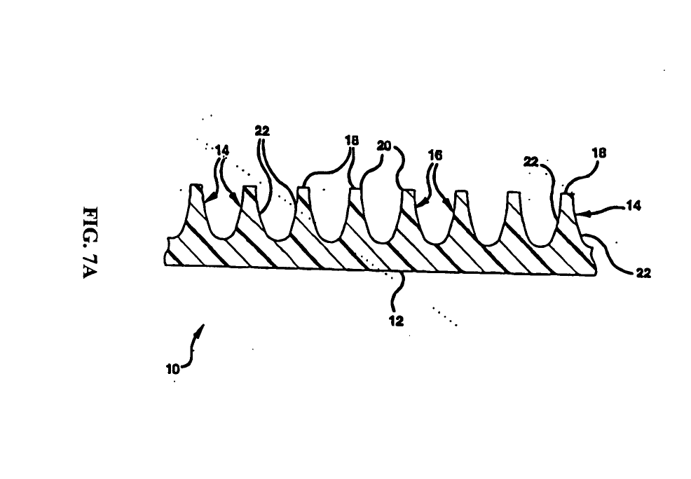

Referring to FIG. 6, the microabrader device 10 of the invention includes a substantially planar body or abrading surface support 12 having a plurality of microprotrusions 14 extending from the bottom surface of the support. The support generally has a thickness sufficient to allow attachment of the surface to the base of the microabrader device thereby allowing the device to be handled easily as shown in FIGS. 4B, 5A and 5B. Alternatively, a differing handle or gripping device can be attached to or be integral with the top surface of the abrading surface support 12. The dimensions of the abrading surface support 12 can vary depending on the length of the microprotrusions, the number of microprotrusions in a given area and the amount of the formulation to be administered to the patient. Typically, the abrading surface support 12 has a surface area of about 1 to 4 cm². In preferred embodiments, the abrading surface support 12 has a surface area of about 1 cm².

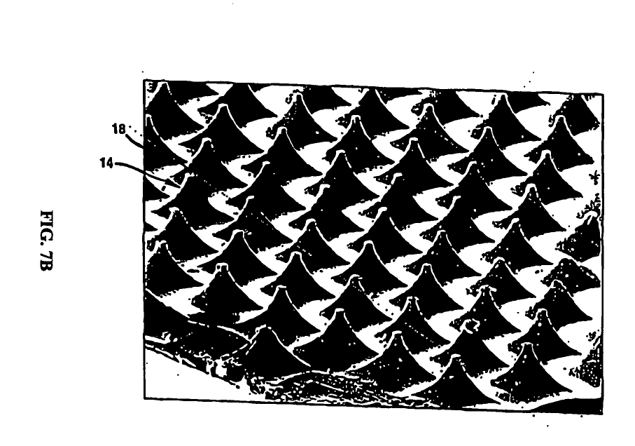

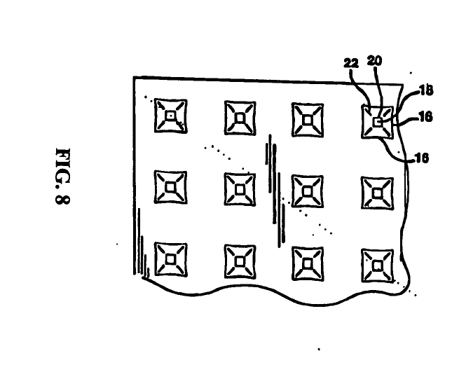

As shown in FIGS. 6, 7A, 7B and 8, the microprotrusions 14 project from the surface of the abrading surface support 12 and are substantially perpendicular to the plane of the abrading surface support 12. The microprotrusions in the illustrated embodiment are arranged in a plurality of rows and columns and are preferably spaced apart a uniform distance. The microprotrusions 14 have a generally pyramid shape with sides 16 extending to a tip 18. The sides 16 as shown have a generally concave profile when viewed in cross-section and form a curved surface extending from the abrading surface support 12 to the tip 18. In the embodiment illustrated, the microprotrusions are formed by four sides 16 of substantially equal shape and dimension. As shown in FIGS. 7A and 8, each of the sides 16 of the microprotrusions 14 have opposite side edges contiguous with an adjacent side and form a scraping edge 22 extending outward from the abrading surface support 12. The scraping edges 22 define a generally triangular or trapezoidal scraping surface corresponding to the shape of the side 16. In further embodiments, the microprotrusions 14 can be formed with fewer or more sides.

The microprotrusions 14 preferably terminate at blunt tips 18. Generally, the tip 18 is substantially flat and parallel to the support 14. When the tips are flat, the total length of the microprotrusions do not penetrate the skin; thus, the length of the microprotrusions is greater than the total depth to which said microprotrusions penetrate said skin. The tip 18 preferably forms a well defined, sharp edge 20 where it meets the sides 16. The edge 20 extends substantially parallel to the abrading surface support 12 and defines a further scraping edge. In further embodiments, the edge 20 can be slightly rounded to form a smooth transition from the sides 16 to the tip 18. Preferably, the microprotrusions are frustoconical or frustopyramidal in shape.

The microabrader device 10 and the microprotrusions can be made from a plastic material that is non-reactive with the substance being administered. A non-inclusive list of suitable plastic materials include, for example, polyethylene, polypropylene, polyamides, polystyrenes, polyesters, and polycarbonates as known in the art. Alternatively, the microprotrusions can be made from a metal such as stainless steel, tungsten steel, alloys of nickel, molybdenum, chromium, cobalt, titanium, and alloys thereof, or other materials such as silicon, ceramics and glass polymers. Metal microprotrusions can be manufactured using various techniques similar to photolithographic etching of a silicon wafer or micromachining using a diamond tipped mill as known in the art. The microprotrusions can also be manufactured by photolithographic etching of a silicon wafer using standard techniques as are known in the art. They can also be manufactured in plastic via an injection molding process, as described for example in U.S. Pat. No. 6,899,838, which is hereby incorporated by reference.

The length and thickness of the microprotrusions are selected based on the particular substance being administered and the thickness of the stratum corneum in the location where the device is to be applied. Preferably, the microprotrusions penetrate the stratum corneum substantially without piercing or passing through the stratum corneum. The microprotrusions can have a length up to about 500 microns. Suitable microprotrusions have a length of about 50 to 500 microns. Preferably, the microprotrusions have a length of about 50 to about 300 microns, and more preferably in the range of about 150 to 250 microns, with 180 to 220 microns most preferred. The microprotrusions in the illustrated embodiment have a generally pyramidal shape and are perpendicular to the plane of the device. These shapes have particular advantages in insuring that abrasion occurs to the desired depth. In preferred embodiments, the microprotrusions are solid members. In alternative embodiments, the microprotrusions can be hollow.

As shown in FIGS. 5 and 8, the microprotrusions are preferably spaced apart uniformly in rows and columns to form an array for contacting the skin and penetrating the stratum corneum during abrasion. The spacing between the microprotrusions can be varied depending on the substance being administered either on the surface of the skin or within the tissue of the skin. Typically, the rows of microprotrusions are spaced to provide a density of about 2 to about 10 per millimeter (mm). Generally, the rows or columns are spaced apart a distance substantially equal to the spacing of the microprotrusions in the array to provide a microprotrusion density of about 4 to about 100 microprotrusions per mm². In another embodiment, the microprotrusions may be arranged in a circular pattern. In yet another embodiment, the microprotrusions may be arranged in a random pattern. When arranged in columns and rows, the distance between the centers of the microprotrusions is preferably at least twice the length of the microprotrusions. In one preferred embodiment, the distance between the centers of the microprotrusions is twice the length of the microprotrusions 110 microns. Wider spacings are also included, up to 3, 4, 5 and greater multiples of the length of the microprotrusions. In addition, as noted above, the configuration of the microprotrusions can be such, that the height to the microprotrusions can be greater than the depth into the skin those protrusions will penetrate.

The flat upper surface of the frustoconical or frustopyramidal microprotrusions is generally 10 to 100, preferably 30-70, and most preferably 35-50 microns in width.

The method of preparing a delivery site on the skin places the microabrader against the skin 28 of the patient in the desired location. The microabrader is gently pressed against the skin and then moved over or across the skin. The length of the stroke of the microabrader can vary depending on the desired size of the delivery site, defined by the delivery area desired. The dimensions of the delivery site are selected to accomplish the intended result and can vary depending on the substance, and the form of the substance, being delivered. For example, the delivery site can cover a large area for treating a rash or a skin disease. Generally, the microabrader is moved about 2 to 15 centimeters (cm). In some embodiments of the invention, the microabrader is moved to produce an abraded site having a surface area of about 4 cm² to about 300 cm².

The microabrader is then lifted from the skin to expose the abraded area and a suitable delivery device, patch or topical formulation may be applied to the abraded area. Alternatively, the substance to be administered may be applied to the surface of the skin either before, or simultaneously with abrasion.

The extent of the abrasion of the stratum corneum is dependent on the pressure applied during movement and the number of repetitions with the microabrader. In one embodiment, the microabrader is lifted from the skin after making the first pass and placed back onto the starting position in substantially the same place and position. The microabrader is then moved a second time in the same direction and for the same distance. In another embodiment, the microabrader is moved repetitively across the same site in alternating direction without being lifted from the skin after making the first pass. Generally, two or more passes are made with the microabrader.

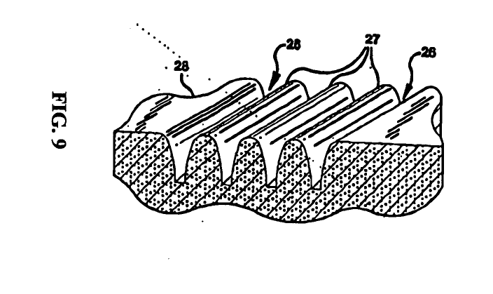

In further embodiments, the microabrader can be swiped back and forth, in the same direction only, in a grid-like pattern, a circular pattern, or in some other pattern for a time sufficient to abrade the stratum corneum a suitable depth to enhance the delivery of the desired substance. The linear movement of the microabrader across the skin 28 in one direction removes some of the tissue to form grooves 26, separated by peaks 27 in the skin 28 corresponding to substantially each row of microprotrusions as shown in FIG. 9. The edges 20, 22 and the blunt tip 18 of the microprotrusions provide a scraping or abrading action to remove a portion of the stratum corneum to form a groove or furrow in the skin rather than a simple cutting action. The edges 20 of the blunt tips 18 of the microprotrusions 14 scrape and remove some of the tissue at the bottom of the grooves 26 and allows them to remain open, thereby allowing the substance to enter the grooves for absorption by the body. Preferably, the microprotrusions 14 are of sufficient length to penetrate the stratum corneum and to form grooves 26 having sufficient depth to allow absorption of the substance applied to the abraded area without inducing pain or unnecessary discomfort to the patient. Preferably, the grooves 26 do not pierce but can extend through the stratum corneum. The edges 22 of the pyramid shaped microprotrusions 14 form scraping edges that extend from the abrading surface support 12 to the tip 18. The edges 22 adjacent the abrading surface support 12 form scraping surfaces between the microprotrusions which scrape and abrade the peaks 27 formed by the skin between the grooves 26. The peaks 27 formed between the grooves generally are abraded slightly.

Any device known in the art for disruption of the stratum corneum by abrasion can be used in the methods of the invention. These include for example, microelectromechanical (MEMS) devices with arrays of short microneedles or microprotrusions, sandpaper-like devices, scrapers and the like.

The actual method by which the epidermal immunogenic compositions of the invention are targeted to the epidermal space is not critical as long as it penetrates the skin of a subject to the desired targeted depth. The microabraiders discussed within initially deposit the inventive formulations to a skin depth of 0.0 to 0.025 mm and preferably not exceeding the stratum corneum.

5.4 Determination of Therapeutic Efficacy

The invention encompasses methods for determining the efficacy of compositions of the invention using any standard method known in the art or described herein. Assays for determining the efficacy of the compositions of the invention may be in vitro based assays or in vivo based assays, including animal based assays. In some embodiments, the invention encompasses detecting and/or quantitating a humoral immune response against the antigenic or immunogenic agent of a composition of the invention in a sample, e.g., serum, obtained from a subject who has been administered an immunogenic composition of the invention. Preferably, the humoral immune response of the compositions of the invention are compared to a control sample obtained from the same subject, who has been administered a control formulation, e.g., a formulation which simply comprises of the antigenic or immunogenic agent.

In some embodiments, the invention encompasses detecting and/or quantitating a humoral immune response against the antigenic or immunogenic agent of the immunogenic composition of this invention in a sample, e.g., serum, obtained from a subject who has been administered an immunogenic composition of this invention. The humoral immune response of the immunogenic composition of this invention is compared to a control sample obtained from the same subject, who has been administered a control formulation, e.g., a formulation which simply comprises of the antigenic or immunogenic agent.

Assays for measuring humoral immune response are well known in the art, e.g., see, Coligan et al., (eds.), 1997, Current Protocols in Immunology, John Wiley and Sons, Inc., Section 2.1, which is incorporated by reference in its entirety. A humoral immune response may be detected and/or quantitated using standard methods known in the art including, but not limited to, an ELISA assay. The humoral immune response may be measured by detecting and/or quantitating the relative amount of an antibody which specifically recognizes an antigenic or immunogenic agent in the sera of a subject who has been treated with an immunogenic composition of this invention relative to the amount of the antibody in an untreated subject. ELISA assays can be used to determine total antibody titers in a sample obtained from a subject treated with a composition of the invention. In other embodiments, ELISA assays may be used to determine the level of isotype specific antibodies using methods known in the art.

ELISA based assays comprise preparing an antigen, coating the well of a 96 well microtiter plate with the antigen, adding an antibody specific to the antigen conjugated to a detectable compound such as an enzymatic substrate (e.g., horseradish peroxidase or alkaline phosphatase) to the well and incubating for a period of time, and detecting the presence of the antigen. In an ELISA assay, the antibody does not have to be conjugated to a detectable compound; instead, a second antibody (which recognizes the first antibody) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 1.2.1, the entirety of which is incorporated herein by reference.

In the cases where the immunogenic composition comprises an influenza antigen, any method known in the art for the detection and/or quantitation of an antibody response against an influenza antigen is encompassed within the methods of the invention. An exemplary method for determining an influenza antigen directed antibody response may comprise the following: an influenza antigen is used to coat a microtiter plate (Nunc plate); sera from a subject treated with an influenza vaccine formulation of the invention is added to the plate; antisera is added to the plate and incubated for a sufficient time to allow a complex to be formed, i.e., a complex between an antibody in the sera and the antisera. The complex is then detected using standard methods in the art. For exemplary assays for measuring an influenza specific antibody response, see, e.g., Newman et al., 1997, Mechanism of Aging & Development, 93: 189-203; Katz et al., 2000, Vaccine, 18: 2177-87; Todd et al., (Brown and Haaheim, eds.), 1998 in Modulation of the Immune Response to Vaccine Antigens, Dev. Biol. Stand. Basel, Karger, 92: 341-51; Kendal et al., 1982, in Concepts and Procedures for Laboratory-based Influenza Surveillance, Atlanta: CDC, B17-35; Rowe et al., 1999, J. Clin. Micro. 37: 937-43; Todd et al., 1997, Vaccine 15: 564-70; WHO Collaborating Centers for Reference and Research on Influenza, in Concepts and Procedures for Laboratory-based Influenza Surveillance, 1982, p. B-23; all of which are incorporated herein by reference in their entirety.

In some specific embodiments, the following assay method is used for influenza antigens. An influenza antigen (e.g., Influenza APR834 from Charles River SPAFAS) is coated on a 96-well microtiter plate. Typically, the coating solution consists of influenza protein in carbonate buffer. The coating antigen is exposed to the plate for one hour at 37° C. The coating solution is discarded and replaced with a blocking solution, typically consists of phosphate buffered saline with Tween-20 (PBS-TW20) and non-fat dry milk. The blocking solution is exposed to the plate surface for two hours at 37° C., and then discarded. Plate surfaces are washed twice with PBS-TW20, and sera from the test and control groups of subjects are added. The sera from all subjects within a particular group is pooled and assayed at a dilution of, for example, 1:123 or 1:370. The primary antibody is incubated on the coated and blocked plates for an hour, and the plates are washed multiple times with PBS-TW20. A cocktail of anti-mouse horseradish conjugate pool is added at a dilution of, for example, 1:15,000. The horseradish peroxidase secondary antibody cocktail is incubated on the plates for an hour at 37° C., and the plates washed multiple times. A TMB substrate is then added for color development. The color is allowed to develop for 30 minutes in dark and stopped by adding, for example, 0.5 molar sulfuric acid. The plates are read at 450 nm on a plate reader.

Furthermore, when the immunogenic composition comprises an influenza antigen, any method known in the art for the detection and/or quantitation levels of antibody with hemagglutination activity are encompassed within the invention. The hemagglutination inhibition assays are based on the ability of influenza viruses to agglutinate erythrocytes and the ability of specific HA antibodies to inhibit agglutination. Any of the hemagglutination inhibition assays known in the art are encompassed within the methods of the inventions, such as those disclosed in Newman et al., 1997, Mechanism of Aging & Development, 93: 189-203; Kendal et al., 1982, in Concepts and Procedures for Laboratory-based Influenza Surveillance, Atlanta: CDC, B17-35; all of which are incorporated herein by reference in their entirety.

An exemplary hemagglutination inhibition assay comprises the following: sera from subjects treated with an immunogenic composition of the invention containing an influenza antigen are added to microtitre plates; HI-antigenic preparation containing 8 HA units is added to the plates; the mixture is mixed well by gently tapping the plates, and incubated for about 1 hour at 4° C.; erythrocyte suspension, e.g., 0.5% chicken erythrocytes, is added to the micotitre plate and the contents are mixed well by gently tapping the plates; the plates are further incubated at 4° C. until the cell control shows the button of normal settling (controls only contains PBS). Preferably, the serum samples are treated with inhibitors, such as neuraminidase or potassium periodate, to prevent non-specific inhibition of agglutination by serum factors. The HI titre is defined as the dilution factor of the highest dilution of serum that completely inhibits hemagglutination. This is determined by tilting the plates and observing the tear shaped streaming of cells that flow at the same rate as control cells.

The invention encompasses methods for determining the efficacy of the compositions of the invention by measuring cell-mediate immune response. Methods for measuring cell-mediated immune response are known to one skilled in the art and encompassed within the invention. In some embodiments, a T cell immune response may be measured for quantitating the immune response in a subject, for example, by measuring cytokine production using common methods known to one skilled in the art including, but not limited to, ELISA from tissue culture supernatants, flow cytometry based intracellular cytokine staining of cells ex vivo or after an in vitro culture period, and cytokine bead array flow cytometry based assay. In yet other embodiments, the invention encompasses measuring T cell specific responses using common methods known in the art including, but not limited to, chromium based release assay, flow cytometry based tetramer or dimer staining assay using known CTL epitopes.

5.5 Prophylactic and Therapeutic Uses

The invention provides methods of treatment and prophylaxis which involve administering an composition of the invention to a subject, preferably a mammal, and most preferably a human for treating, managing or ameliorating symptoms associated with a disease or disorder, especially an infectious disease or cancer. The subject is preferably a mammal such as a non-primate, e.g., cow, pig, horse, cat, dog, rat, and a primate, e.g., a monkey such as a Cynomolgous monkey and a human. In a preferred embodiment, the subject is a human. Preferably, the composition of the invention is a vaccine compositions.

The invention encompasses a method for eliciting an enhanced immune response in a subject comprising intradermal delivery of a single dose of am immunogenic composition of the invention to a subject, preferably a human, in combination with one or more excipients. In some embodiments, the invention encompasses one or more booster immunizations. The immunogenic composition of the invention is particularly effective in stimulating and/or upregulating an antibody response to a level greater than that seen in conventional immunogenic compositions and administration schedules. For example, an immunogenic composition of the invention may lead to an antibody response comprising generations of one or more antibody classes, such as IgM, IgG, and/or IgA. Most preferably, the immunogenic compositions of the invention stimulate a systemic immune response that protects the subject from at least one pathogen. The immunogenic compositions of the invention may provide systemic, local, or mucosal immunity or a combination thereof.

5.5.1 Target Diseases

The invention encompasses intradermal delivery systems to treat and/or prevent an infectious disease in a subject, preferably a human. Infectious diseases that can be treated or prevented by the methods of the present invention are caused by infectious agents including, but not limited to, viruses, bacteria, fungi protozoa, helminths, and parasites.

Examples of viruses that have been found in humans and can be treated by the delivery systems of the invention include, but are not limited to, Retroviridae (e.g., human immunodeficiency viruses, such as HIV-1 (also referred to as HTLV-III, LAV or HTLV-III/LAV, or HIV-III; and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (e.g., hemorrhagic fever viruses); Reoviridae (e.g., reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted, e.g., Hepatitis C); Norwalk and related viruses, and astroviruses.

Retroviruses that results in infectious diseases in animals and humans and can be treated and/or prevented using the delivery systems and methods of the invention include both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of RNA viruses that are antigenic or immunogenic in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses), the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A (many human subtypes), Swine influenza virus, and Avian and Equine Influenza viruses, influenza type B (many human subtypes), and influenza type C (possible separate genus)); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigenic or immunogenic in vertebrate animals include, but are not limited to: the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus), the Beta-herpes viruses (Human cytomegalovirus and cytomegaloviruses of swine, monkeys and rodents), the gamma-herpesviruses (Epstein-Barr virus (EBV), Marek's disease virus, Herpes saimiri, Herpes virus ateles, Herpes virus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species), the genus Aviadenovirus (Avian adenoviruses), and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents.

Bacterial infections or diseases that can be treated or prevented by the methods of the present invention are caused by bacteria including, but not limited to, bacteria that have an intracellular stage in its life cycle, such as mycobacteria (e.g., Mycobacteria tuberculosis, M. bovis, M. avium, M. leprae, or M. africanum), rickettsia, mycoplasma, chlamydia, and legionella. Other examples of bacterial infections contemplated include, but are not limited to, infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, Neisseria species. Specific examples of infectious bacteria include, but are not limited to: Helicobacter pyloris, Borelia burgdorferi, Legionella pneumophilia, Mycobacteria tuberculosis, M. avium, M. intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, corynebacterium diphtheriae, Erysipelothrix rhusiopathiae, Clostridium perfringers, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelli.

Fungal diseases that can be treated or prevented by the methods of the present invention include, but are not limited to, aspergilliosis, crytococcosis, sporotrichosis, coccidioidomycosis, paracoccidioidomycosis, histoplasmosis, blastomycosis, zygomycosis, and candidiasis.

Parasitic diseases that can be treated or prevented by the methods of the present invention include, but are not limited to, amebiasis, malaria, leishmania, coccidia, giardiasis, cryptosporidiosis, toxoplasmosis, and trypanosomiasis. Also encompassed are infections by various worms such as, but not limited to, ascariasis, ancylostomiasis, trichuriasis, strongyloidiasis, toxoccariasis, trichinosis, onchocerciasis. filaria, and dirofilariasis. Also encompassed are infections by various flukes such as, but not limited to, schistosomiasis, paragonimiasis, and clonorchiasis. Parasites that cause these diseases can be classified based on whether they are intracellular or extracellular. An “intracellular parasite,” as used herein, is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite,” as used herein, is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites.” These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp.

The invention also encompasses immunogenic compositions to treat and/or prevent cancers, including, but not limited to, neoplasms, tumors, metastases, or any disease or disorder characterized by uncontrolled cell growth. For example, but not by way of limitation, cancers and tumors associated with the cancer and tumor antigens listed supra in Section 5.1.2 may be treated and/or prevented using the immunogenic compositions of the invention.

5.6 Kits

The invention further comprises kits comprising an intradermal administration device and a composition of the invention as described herein. In some embodiments, the invention also provides a pharmaceutical pack or kit comprising a composition of the invention. In a specific embodiment, the invention provides a kit comprising, one or more containers filled with one or more of the components of the compositions of the invention, e.g., an anitgenic or immunogenic agent, an adjuvant compound. In another specific embodiment, the kit comprises two containers, one containing an antigenic or immunogenic agent, and the other containing the adjuvant. Associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLES

Aspects of this invention are illustrated by the following non-limiting examples.

6.1 Immune Response from the Administration of Fluzone

6.1.1 Preparation of Inoculum

Prior to preparation of various formulations, the pH of all adjuvant stock solutions were checked for a neutral pH, i.e., 7.0-7.4. The pH of the solutions was adjusted to neutral as necessary using dilute HCl or NaOH. All adjuvant stock solutions were sterile filtered through a 0.2 micron Gelman Acrodisc PF syringe filter #4187.

Inoculums were prepared by adding 175 μl of Aventis Fluzone YR 02/03 and the adjuvant compounds at varying concentrations. Hanks Buffered Saline Solution (HBSS) was used to bring the final volume to 700 μl. A control inoculum was prepared by adding HBSS to 175 μl of Fluzone to yield a final volume of 700 μl. Each animal was inoculated by using 100 μl of the prepared inoculums. For non-immune control, the animal received 100 μl of HBSS.

6.1.2 Administration

Inoculum was injected into Balb/c mice within an hour of preparation. The mice used for inoculation were obtained from Charles River Laboratories and were between 4 and 8 weeks of age. Balb/c mice used in the study had an average weight of about 20 grams. The mice were dry-shaved just prior to injection using a Conair Electric shaver. Approximately 15 minutes prior to the inoculation, each mouse received an intraperitoneal injection of ketamine/xylazine/acepromazine cocktail for sedation. The lower to mid back region was used for injection of flu immunogen.

Each inoculum was drawn up into a 1 ml latex free syringe (BD Cat. 309628) fixed with a 20 g needle (BD Cat. 305179). After the syringe was loaded the 20 g needle was replaced with a 30 g needle for intradermal (ID) administration. The Mantoux method of ID administration was used whereby the skin is tightly pulled and the needle is approached at the most shallow possible angle with the bevel up. The injection volume was pushed in slowly over 5-10 seconds forming the typical “bleb” and then the needle was slowly removed. To prevent the spill over of the inoculum into surrounding tissue space, only one injection was employed and the injection volume was kept at 100 μl. Animals were monitored for local and systematic indications of toxicity immediately after administration at 24 hours after the inoculation, and again at three weeks. No signs of local or systematic toxicity were observed in animals.

6.1.3. Assays

Antibody response to Fluzone was measured by coating an influenza antigen (Influenza APR834, purified/inactivated at 2 mg/ml from Charles River SPAFAS) on a microtiter plate (96-well Nunc Immuno-Plate® with MaxiSorp® surface). The coating solution was 3.8 μg/ml of influenza protein in carbonate buffer (Sigma Chemical Co. Cat. C3041). The coating antigen was exposed to the Nunc plate for one hour at 37° C. The coating solution was discarded and replaced with a blocking solution (phosphate buffered saline with Tween 20 (PBS-TW20); Sigma Chemical Co. Cat. P-3563) and 5% w/v nonfat dry milk. The blocking solution was exposed to the plate surface for two hours at 37° C. The blocking solution was subsequently discarded.

Plate surfaces were washed twice with PBS-TW20 and sera from test/control groups were added. Sera were collected 21 days after immunization. Sera from all animals in each particular test or control group were pooled and used for the assays. The pooled serum was assayed at 1:123 and 1:370 dilutions.

The primary antibody was incubated on the coated and blocked plates for an hour, and the plates were washed three time with PBS-TW20. A cocktail of anti-mouse horseradish peroxidase conjugate pool, which consisted of Sigma A4416, Southern Biotech 1090-05, Southern Biotech 1070-05, Southern Biotech 1080-05 and Southern Biotech 1100-05, was added. All conjugates were present at a 1:15,000 dilution in the final cocktail. The horseradish peroxidase secondary antibody cocktail was incubated on the plates for an hour at 37° C. The plates were then washed three times with PBS-TW20.

For color development, Sigma T-8665, a TMB substrate, was added, and the color was allowed to develop for 30 minutes in the dark. Color development was stopped by the addition of 0.5 molar sulfuric acid, and the plates were read at 450 nm on a TECAN SUNRISE plate reader.

6.1.4 Results

As shown in Table 1, the inoculums that contained certain adjuvant compounds resulted in a greater immune response (as indicated by X) as compared to the inoculums that contained Fluzone alone, or the non-immune inoculums. This result clearly shows that these compounds can act as adjuvants when administered together with an antigenic or immunogenic agent into the subject's intradermal compartment.

6.2 Immune Response Form the Administration of a Plasmid DNA Comprising a Sequence that Codes Flu Hemagglutinin

6.2.1 Preparation of Inoculum

Prior to preparation of various formulations, the pH of all adjuvant compound stock solutions were checked for a neutral pH, i.e., 7.0-7.4. The pH of the solutions was adjusted to neutral as necessary using dilute HCl or NaOH. All adjuvant stocks were sterile filtered through a 0.2 micron Gelman Acrodisc PF syringe filter #4187.

Inoculums were prepared by adding 350 μg of a plasmid DNA comprising a sequence that encodes flu hemagglutinin (pDNA-HA) and the adjuvant compound at varying concentrations. HBSS was used to bring the final volume to 700 μl. A control inoculum was prepared by adding HBSS to 350 μg of pDNA-HA to yield a final volume of 700 μl. Each animal was inoculated by using 100 μl of the prepared inoculums. For non-immune control, the animal received 100 μl of HBSS.

6.2.2 Administration

Inoculum was injected into Balb/c mice within an hour of preparation. The mice used for inoculation were obtained from Charles River Laboratories and were between 4 and 8 weeks of age. The mice were dry-shaved just prior to injection using a Conair Electric shaver. Approximately 15 minutes prior to the inoculation, each mouse received an intraperitoneal injection of ketamine/xylazine/acepromazine cocktail for sedation. The lower to mid back region was used for injection.

Each inoculum was drawn up into a 1 ml latex free syringe (BD Cat. 309628) fixed with a 20 g needle (BD Cat. 305179). After the syringe was loaded the 20 g needle was replaced with a 30 g needle for intradermal (ID) administration. The Mantoux method of ID administration was used whereby the skin is tightly pulled and the needle is approached at the most shallow possible angle with the bevel up. The injection volume was pushed in slowly over 5-10 seconds forming the typical “bleb” and then the needle was slowly removed. To prevent the spill over of the inoculum into surrounding tissue space, only one injection was employed and the injection volume was kept at 100 μl.

Animals were monitored for local and systematic indications of toxicity immediately after administration at 24 hours after the inoculation, and again at three weeks. No signs of local or systematic toxicity were observed in animals.

6.2.3 Assays

Antibody response to the various inoculums that comprise pDNA-HA was measured by coating an influenza antigen (Influenza APR834, purified/inactivated at 2 mg/ml from Charles River SPAFAS) on a microtiter plate (96-well Nunc Immuno-Plate® with MaxiSorps surface). The coating solution was 3.8 μg/ml of influenza protein in carbonate buffer (Sigma Chemical Co. Cat. C3041). The coating antigen was exposed to the Nunc plate for one hour at 37° C. The coating solution was discarded and replaced with a blocking solution (PBS-TW20) and 5% w/v nonfat dry milk. The blocking solution was exposed to the plate surface for two hours at 37° C. The blocking solution was subsequently discarded.

Plate surfaces were washed twice with PBS-TW20 and sera from test/control groups were added. Sera were collected 21 days after immunization. Sera from animals in same test or control group were pooled and used for the assays. The pooled serum was assayed at 1:123 and 1:370 dilutions.

The primary antibody was incubated on the coated and blocked plates for an hour, and the plates were washed three time with PBS-TW20. A cocktail of anti-mouse horseradish peroxidase conjugate pool, which consisted of Sigma A4416, Southern Biotech 1090-05, Southern Biotech 1070-05, Southern Biotech 1080-05 and Southern Biotech 1100-05, was added. All conjugates were present at a 1:15,000 dilution in the final cocktail. The horseradish peroxidase secondary antibody cocktail was incubated on the plates for an hour at 37° C. The plates were then washed three times with PBS-TW20.

For color development, Sigma T-8665, a TMB substrate, was added, and the color was allowed to develop for 30 minutes in the dark. Color development was stopped by the addition of 0.5 molar sulfuric acid, and the plates were read at 450 nm on a TECAN SUNRISE plate reader.

6.2.4 Results

As shown in Table 1, inoculums that contain certain adjuvant compounds resulted in an increased immune response from the animals (as denoted by X) as compared to the inoculums that contained pDNA-HA alone, or non-immune inoculums. This result clearly shows that these compounds can act as adjuvants when administered together with an antigenic or immunogenic agent into the intradermal compartment.

TABLE 1
Increased Immune Response By Various Adjuvant Compounds

| Compound | Fluzone | pDNA-HA |
|---|---|---|
| Aluminum Phosphate | X | X |
| Calcium Phosphate | | X |
| Complement Factor C3d | X | X |
| CpG | X | |
| Interferon-gamma | X | X |
| IL-2 | | X |
| IL-4 | | X |
| IL-6 | X | X |
| IL-7 | X | X |
| IL-12 | X | X |
| IL-15 | | X |
| MIP-3a | | X |
| Quil-A | X | |
| Immther ™ | | X |
| MPL-A | | X |
| Mannon | | X |
| Melanonin Peptide 946 | X | X |
| Neutrophil Chemo-attractant | X | X |
| Peptide | | |
| Elastin Repeating Peptide | X | X |

Results of ELISA assays are summarized in Tables below. The values below reflect relative responses (flu specific antibody) to the various formulations. The assay was performed using a solid phase ELISA performed as described above. Serum samples were collected 21 days after immunization and the animals only received one immunization.

Table. 2A-D: Serum Titers to Influenza Antigen

(ELISA Signals at 1:123 Dilution)

TABLE 2A
STUDY 1: DISRUPTED VIRION IMMUNOGEN

| Test or Control | | Disr. Virion | Disr. Virion + | Disr. Virion + | Disr. Virion + | Disr. Virion + C3d |
|---|---|---|---|---|---|---|
| Group | Non-Immune | Alone | IL-6 Adjuvant | IL-7 Adjuvant | IL-12 Adjuvant | Pep. Adjuvant |
| Avg signal | 0.11 | 0.665 | 0.967 | 1.284 | 0.852 | 0.905 |
| Stdev | 0.014 | 0.029 | 0.077 | 0.009 | 0.035 | 0.044 |

TABLE 2B
Study 2: Disrupted Virion Immunogen

| | | | | | Disr. Virion + |
|---|---|---|---|---|---|
| | | | | | Neutrophil. |
| | | | Disr. Virion + | Disr. Virion + | Chemoatt. |
| Test or Control | | Disr. Virion | Elastin Repeating | Melanonin | Peptide |
| Group | Non-Immune | Alone | Peptide Adjuvant | Peptide Adjuvant | Adjuvant |
| Avg signal | 0.135 | 0.398 | 0.598 | 0.656 | 0.718 |
| Stdev | 0.001 | 0.005 | 0.025 | 0.037 | 0.018 |

TABLE 2C
Study 3: Disr Virion Immunogen

| Test or | | | Disr. Virion + | Disr. Virion + |
|---|---|---|---|---|
| Control | | Disr. Virion | Saponin | CpG |
| Group | Non-Immune | Alone | Adjuvant | Adjuvant |
| Avg signal | 0.18 | 0.704 | 2.6463 | 1.273 |
| Stdev | 0.005 | 0.010 | 0.111 | 0.012 |

TABLE 2D
Study 4: Disr Virion Immunogen

| | | | Disr. Virion + |
|---|---|---|---|
| Test or | | | Aluminum |
| Control | | Disr. Virion | Phosphate |
| Group | Non-Immune | Alone | Adjuvant |
| Avg signal | 0.110 | 0.600 | 1.349 |
| Stdev | 0.001 | 0.050 | 0.243 |

Table 3A-B: Serum Titers to Influenza Antigen

(ELISA Signals at 1:123 Dilution)

TABLE 3A
STUDY 5: PLASMID DNA-HA IMMUNOGEN

| Test or | | | | | | |
|---|---|---|---|---|---|---|
| Control | Non- | pDNA-HA | pDNA-HA + | pDNA-HA + | pDNA-HA + | pDNA-HA + |
| Group | Immune | Alone | IL-2 | IL-4 | IL-6 | IL-7 |
| Avg signal | 0.148 | 0.297 | 0.736 | 0.956 | 1.687 | 0.417 |
| Stdev | 0.031 | 0.005 | 0.012 | 0.043 | 0.072 | 0.011 |

TABLE 3B
Study 6: Plasmid DNA-HA Immunogen

| Test or | | | |
|---|---|---|---|
| Control | Non- | pDNA-HA | pDNA-HA + Mannon |
| Group | Immune | Alone | Adjuvant |
| Avg signal | 0.258 | 0.299 | 0.724 |
| Stdev | 0.014 | 0.012 | 0.004 |

Table 4A-C: Elisa Signals at 1:370 Dilution

TABLE 4A
Study 7: Plasmid DNA-HA Immunogen

| Test or | | | pDNA-HA + C3d | pDNA-HA + |
|---|---|---|---|---|
| Control | | pDNA-HA | Peptide | IL-12 |
| Group | Non-Immune | Alone | Adjuvant | Adjuvant |
| Avg signal | 0.141 | 0.224 | 0.730 | 1.650 |
| Stdev | 0.028 | 0.024 | 0.044 | 0.034 |

TABLE 4B
STUDY 8: PLASMID DNA-HA IMMUNOGEN

| | | | | | pDNA-HA + | pDNA-HA + |
|---|---|---|---|---|---|---|
| Test or | | | pDNA-HA + | pDNA-HA + | Calcium | Aluminum |
| Control | Non- | pDNA-HA | IFN-Y | IL-15 | Phosphate | Phosphate |
| Group | Immune | Alone | Adjuvant | Adjuvant | Adjuvant | Adjuvant |
| Avg signal | 0.126 | 0.341 | 0.694 | 0.617 | 0.975 | 1.298 |
| Stdev | 0.008 | 0.019 | 0.016 | 0.044 | 0.099 | 0.131 |

TABLE 4C
STUDY 9: PLASMID DNA-HA IMMUNOGEN

| | | | | | | pDNA-HA + | pDNA-HA + | |
|---|---|---|---|---|---|---|---|---|
| | | | | | | Elastin | Neutrophil. | pDNA-HA + |
| Test or | | | pDNA-HA + | pDNA-HA + | pDNA-HA + | Repeating | Chemoatt. | Melanonin |
| Control | Non- | pDNA-HA | MPL-A | MIP-3a | ImmTher ™ | Peptide | Peptide | Peptide |
| Group | Immune | Alone | Adjuvant | Adjuvant | Adjuvant | Adjuvant | Adjuvant | Adjuvant |
| Avg signal | 0.162 | 0.255 | 0.995 | 1.007 | 0.693 | 0.775 | 0.849 | 0.986 |
| Stdev | 0.044 | 0.015 | 0.085 | 0.029 | 0.039 | 0.038 | 0.042 | 0.012 |

All of the references cited herein are incorporated by reference in their entirety. While the invention has been described with respect to the particular embodiments, it will be apparent to those skilled in the art that various changes and modifications may be made without departing from the spirit and scope of the invention as recited by the appended claims.

What is claimed is:

1. A method for administering a vaccine formulation to a subject comprising delivering the vaccine formulation to the intradermal compartment of the subject's skin, wherein the formulation comprises an antigenic or immunogenic agent, and an adjuvant selected from the group consisting of:
a. a mineral salt, wherein the concentration of the mineral salt is from about 0.01% to about 10% sediment or v/v, wherein the formulation is not liposomal or an emulsion;
b. a bacterial or yeast antigen, wherein the concentration of the bacterial or yeast antigen is from about 0.1 μg/mL to 1000 μg/mL or from about 10 ng to 100 μg total adjuvant per dose, wherein the bacterial or yeast antigen is not the heat labile toxin of E. coli;
c. an immunostimulatory oligonucleotide, wherein the concentration of the immunostimulatory oligonucleotide is from about 0.9 μg/mL to 900 μg/mL or from about 90 ng to 90 μg total adjuvant per dose;
d. a saponin, wherein the concentration of the saponin is from about 0.1 μg/mL to about 100 μg/mL or from about 10 ng to 10 μg total adjuvant per dose, provided that the formulation is not liposomal, an emulsion, and wherein the antigenic agent is not fused to a heterologous sequence, excluding immunostimulatory oligonucleotides and AGPs;
e. a serum protein, wherein the concentration of the serum protein is from about 0.1 μg/mL to about 100 μg/mL or from about 10 ng to 10 μg total adjuvant per dose;
f. a mammalian peptide, wherein the concentration of the mammalian peptide is from about 0.1 μg/mL to about 100 μg/mL, or from about 10 ng to 10 μg total adjuvant per dose; and
g. a cytokine other that IL-12, wherein the concentration of the cytokine is from about 0.1 μg/mL to about 100 μg/mL, or from about 10 ng to 10 μg total adjuvant per dose;
wherein the immune response to the antigenic or immunogenic agent is enhanced when the formulation is deposited in the intradermal compartment.

2. The method of claim 1, wherein the vaccine formulation is delivered via a microneedle.

3. The method of claim 2, wherein the microneedle is inserted into the subject's skin at an angle perpendicular to the subject's skin to a depth within the intradermal space.

4. The method of claim 1, wherein the antigenic or immunogenic agent is an influenza subunit antigen, a plasmid DNA containing a sequence encoding hemagglutinin, or a disrupted influenza virion immunogen.

5. The method of claim 1, wherein the adjuvant is a mineral salt and wherein the concentration of the mineral salt is from about 0.01% to about 1% sediment or v/v.

6. The method of claim 1, wherein the adjuvant is a mineral salt and wherein the concentration of the mineral salt is from about 0.01% to about 0.1% sediment of v/v.

7. The method of claim 1, wherein the adjuvant is a bacterial or yeast antigen and wherein the concentration of the bacterial or yeast antigen is from about 0.1 μg/mL to about 100 μg/mL.

8. The method of claim 1, wherein the adjuvant is a bacterial or yeast antigen and wherein the concentration of the bacterial or yeast antigen is from about 0.1 μg/mL to about 10 μg/mL.

9. The method of claim 1, wherein the adjuvant is a bacterial or yeast antigen and wherein the concentration of the bacterial or yeast antigen is from about 0.1 μg/mL to about 1 μg/mL.

10. The method of claim 1, wherein the adjuvant is an immunostimulatory oligonucleotide and wherein the concentration of the immunostimulatory oligonucleotide is from about 0.1 μg/mL to about 100 μg/mL.

11. The method of claim 1, wherein the adjuvant is an immunostimulatory oligonucleotide and wherein the concentration of the immunostimulatory oligonucleotide is from about 1 μg/mL to about 50 μg/mL.

12. The method of claim 1, wherein the adjuvant is an immunostimulatory oligonucleotide and wherein the concentration of the immunostimulatory oligonucleotide is from about 5 μg/mL to about 20 μg/mL.

13. The method of claim 1, wherein the adjuvant is a saponin and wherein the concentration of the saponin is from about 1 μg/mL to about 50 μg/mL.

14. The method of claim 1, wherein the adjuvant is a saponin and wherein the concentration of the saponin is from about 5 μg/mL to 20 μg/mL.

15. The method of claim 1, wherein the adjuvant is a serum protein and wherein the concentration of the serum protein is from about 0.1 μg/mL to about 10 μg/mL.

16. The method of claim 1, wherein the adjuvant is a serum protein and wherein the concentration of the serum protein is from about 0.1 μg/mL to about 1 μg/mL.

17. The method of claim 1, wherein the adjuvant is a mammalian peptide and wherein the concentration of the mammalian peptide is from about 0.1 μg/mL to about 10 μg/mL.

18. The method of claim 1, wherein the adjuvant is a mammalian peptide and wherein the concentration of the mammalian peptide is from about 0.1 μg/mL to about 1 μg/mL.

19. The method of claim 1, wherein the adjuvant is a cytokine and wherein the concentration of the cytokine is from about 0.1 μg/mL to about 10 μg/mL.

20. The method of claim 1, wherein the adjuvant is a cytokine and wherein the concentration of the cytokine is from about 0.1 μg/mL to about 1 μg/mL.

21. A kit for administering a vaccine formulation, said kit comprising: (a) an antigenic or immunogenic agent; (b) a device comprising a microneedle with a length sufficient to penetrate the intrademal space and an outlet at a depth within the intraderaml space; and (c) an adjuvant selected from the group consisting of:
a. a mineral salt, wherein the concentration of the mineral salt is from about 0.01% to about 10% sediment or v/v, wherein the formulation is not liposomal or an emulsion;
b. a bacterial or yeast antigen, wherein the concentration of the bacterial or yeast antigen is from about 0.1 μg/mL to 1000 μg/mL or from about 10 ng to 100 μg total adjuvant per dose, wherein the bacterial or yeast antigen is not the heat labile toxin of E. coli;
c. an immunostimulatory oligonucleotide, wherein the concentration of the immunostimulatory oligonucleotide is from about 0.9 μg/mL to 900 μg/mL or from about 90 ng to 90 μg total adjuvant per dose;
d. a saponin, wherein the concentration of the saponin is from about 0.1 μg/mL to about 100 μg/mL or from about 10 ng to 10 μg total adjuvant per dose, provided that the formulation is not liposomal, an emulsion, and wherein the antigenic agent is not fused to a heterologous sequence, excluding immunostimulatory oligonucleotides and AGPs;
e. a serum protein, wherein the concentration of the serum protein is from about 0.1 μg/mL to about 100 μg/mL or from about 10 ng to 10 μg total adjuvant per dose;
f. a mammalian peptide, wherein the concentration of the mammalian peptide is from about 0.1 μg/mL to about 100 μg/mL, or from about 10 ng to 10 μg total adjuvant per dose; and
g. a cytokine other that IL-12, wherein the concentration of the cytokine is from about 0.1 μg/mL to about 100 μg/mL, or from about 10 ng to 10 μg total adjuvant per dose.

22. The kit of claim 21, wherein the needle is no more than 2 mm in length.

23. The kit of claim 21, wherein the needle is about 300 μm to about 2 mm in length.

24. The kit of claim 21, wherein the needle is about 500 μm to about 1 mm in length.

25. The kit of claim 21, wherein the antigenic or immunogenic agent is an influenza subunit antigen, a sequence encoding hemagglutinin, or a disrupted influenza virion immunogen.
